(19) United States
(12) Patent Application Publication
(10) Pub. No.: US 2022/0213198 A1
(43) Pub. Date: Jul. 7, 2022

(54) METHOD OF TREATMENT

(71) Applicant: The Council of the Queensland Institute of Medical Research, Herston (AU)

(72) Inventors: Michele WL TENG, Brisbane (AU); Juming YAN, Brisbane (AU); Mark J SMYTH, Brisbane (AU)

(21) Appl. No.: 17/612,888

(22) PCT Filed: May 22, 2020
(86) PCT No.: PCT/AU2020/050508

§ 371 Date: Nov. 19, 2021

(30) Foreign Application Priority Data
May 22, 2019 (AU) 2019901742

(51) Int. Cl. C07K 16/28; A61P 35/00

(52) U.S. Cl. CPC C07K 16/2833; C07K 16/2818; C07K 16/2827; C07K 2317/31; C07K 16/2896; C07K 16/2803; A61P 35/00; C07K 16/286

(57) ABSTRACT

The present disclosure relates to methods for the treatment of cancer, methods of increasing the response of tumor cells in a subject to cancer therapy, as well as methods of predicting a positive clinical response to a cancer therapy in a patient.

### CROSS-REFERENCE TO RELATED APPLICATIONS

The present application claims priority from Australian Provisional Patent Application No 2019901742 filed on 22 May 2019, the content of which is incorporated herein by reference in its entirety.

### TECHNICAL FIELD

The present disclosure relates to methods for the treatment of cancer, methods of increasing the response of tumor cells in a subject to cancer therapy, as well as methods of predicting a positive clinical response to a cancer therapy in a patient.

### BACKGROUND

Cancer occurs after cells are genetically altered to proliferate rapidly and indefinitely. The cells which constitute the tumor eventually undergo metaplasia, followed by dysplasia then anaplasia, resulting in a malignant phenotype. This malignancy allows for invasion into the circulation, followed by invasion to a second site for tumorigenesis. Some cancer cells known as circulating tumor cells acquire the ability to penetrate the walls of lymphatic or blood vessels, after which they are able to circulate through the bloodstream to other sites and tissues in the body. After the tumor cells come to rest at another site, they may re-penetrate the vessel and continue to multiply, eventually forming another clinically detectable tumor. This new tumor is known as a metastatic (or secondary) tumor. Metastasis is one of the hallmarks of cancer, distinguishing it from benign tumors. Most cancers can metastasize, although in varying degrees. Treatment and prognosis is determined, to a great extent, by whether or not a cancer remains localized or is at an increased risk of spreading to other locations in the body.

There remains a need for compositions and methods to prevent, inhibit or reduce tumor initiation, promotion, growth and/or metastasis.

### SUMMARY

Mucosal-associated invariant T (MAIT) cells are innate-like T cells that require MHC class I-related protein 1 (MR1) for their development. Although MAIT cells are reported to have immunoregulatory roles in infection and autoimmune diseases, their role in tumor immunity has been unclear. The present disclosure shows that MAIT cells display tumor-promoting function and that blocking MR1, or saturating it with an inhibitory ligand, is a new therapeutic strategy for cancer immunotherapy.

Accordingly, in some embodiments there is provided a method of treating cancer in a subject, the method comprising administering to the subject a compound that inhibits MR1-mediated MAIT cell activation.

In some embodiments, there is provided the use of a compound that inhibits MR1-mediated MAIT cell activation in the manufacture of a medicament for treating cancer in a subject.

In some embodiments, there is provided a compound that inhibits MR1-mediated cell activation for use in the treatment of cancer in a subject.

In some embodiments, there is provided a method of treating cancer in a subject, the method comprising administering a compound that binds to MR1 on tumor cells in the subject.

In some embodiments, there is provided the use of a compound that binds to MR1 on tumor cells in a subject in the manufacture of a medicament for treating cancer in the subject.

In some embodiments, there is provided a compound that binds to MR1 on tumor cells in a subject for use in the treatment of cancer in the subject.

In some embodiments, the compound binds to MR1 and inhibits MR1-mediated MAIT cell activation.

There is further provided a method of increasing the response of tumor cells in a subject to cancer therapy, the method comprising administering to the subject a compound that binds to MR1 on tumor cells in the subject.

In some embodiments, the cancer therapy is cancer immunotherapy or targeted therapy.

In some embodiments, the cancer immunotherapy is selected from antibody therapy, CAR-T cell therapy, immune checkpoint inhibitor therapy, and/or cytokine therapy.

In some embodiments, the compound binds to MR1 and inhibits MR1 signalling of MAIT cells.

In some embodiments, the compound that binds to MR1 may be a ligand that inhibits MR1 signalling of MAIT cells.

In one particular embodiment, the ligand is acetyl 6-formylpterin (Ac-6-FP).

In some embodiments, the compound that binds to MR1 may be an antibody.

In some embodiments, the antibody is a multivalent antibody.

In one particular embodiment the multivalent antibody is a bispecific antibody.

In some embodiments, the multivalent antibody binds to an immune checkpoint molecule, T-cell surface molecule, and/or an NK cell surface molecule.

In some embodiments, the immune checkpoint molecule is selected from PD-1, PD-L1, CTLA-4, A2AR, CD73, B7-H3, B7-H4, BTLA, IDO, KIR, LAG3, NOX2, TIM-3, VISTA, CD39, TIGIT, CD96, CD155, IL23R and SIGLEC7.

In some embodiments, administering a compound that inhibits MR1-mediated MAIT cell activation and/or that binds to MR1 on cancer cells in a subject increases T cell tumor infiltration, NK cell tumor infiltration, T cell activation, and/or NK cell activation in the subject.

In some embodiments, the method comprises administering to the subject a pharmaceutical composition comprising the compound and a pharmaceutically acceptable carrier and/or diluent.

In some embodiments, the method is performed in conjunction with an additional cancer therapy. In some embodiments, the medicament may be administered in conjunction with an additional cancer therapy. In some embodiments, the compound may be administered in conjunction with an additional cancer therapy. The additional cancer therapy may be selected from, for example, radiotherapy, surgery, targeted therapy and/or chemotherapy.

There is further provided a method of determining the likelihood of cancer metastasis in a subject, the method comprising:

(a) detecting the level of expression of MR1 in tumor cells in the subject, and

(b) comparing the level of expression of MR1 in tumor cells in the subject to a reference level of expression of MR1 in cancer cells,

wherein a higher level of expression of MR1 in tumor cells in the subject compared to the reference level of expression of MR1 in tumor cells is indicative of the patient having an increased risk of cancer metastasis.

In some embodiments, the reference level of expression of MR1 in tumor cells is derived from a control sample, a normal reference sample and/or a predetermined level of MR1 expression in tumor cells.

In some embodiments, the method comprises obtaining a sample comprising tumor cells from the subject and detecting the level of expression of MR1 in the tumor cells.

In some embodiments, the method comprises detecting MR1 polypeptide on the cell surface of the tumor cells.

In some embodiments, method comprises contacting the tumor cells with a compound that binds to MR1 and detecting the compound bound to MR1.

In some embodiments, the compound that binds to MR1 is an antibody.

In some embodiments, the method comprises quantitating MR1 mRNA in tumor cells.

There is further provided a method of selecting a patient for treatment with a compound that binds MR1 on tumor cells, the method comprising determining whether MR1 is expressed in tumor cells in the patient, wherein a patient is selected for treatment with a compound that binds MR1 on the basis of MR1 expression in the tumor cells.

There is further provided a method of determining a likelihood of a positive or negative clinical response in a patient to a cancer therapy, the method comprising comparing the level of expression of MR1 in tumor cells in the subject to a reference level of expression of MR1 in tumor cells,

wherein a higher level of expression of MR1 in tumor cells in the subject compared to the reference level of expression of MR1 in tumor cells is indicative of the patient having an increased likelihood of a positive clinical response to the cancer therapy, wherein the cancer therapy comprises administration of compound that inhibits MR1-mediated MAIT cell activation and/or that binds to MR1 on tumor cells in the patient.

In some embodiments, the method comprises detecting the level of expression of MR1 polypeptide on tumor cells in the subject.

There is further provided a method of preventing or reducing the likelihood of cancer metastasis in a subject, the method comprising administering to the subject a compound that binds to MR1 on tumor cells in the subject.

In any of the methods or uses provided herein, the cancer may be selected from the group consisting of: lung cancer, non-small-cell lung carcinoma, small-cell lung carcinoma, fibrosarcoma, colorectal carcinoma and osteosarcoma.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1. Tumor initiation, growth and metastases are suppressed in MR1^{-/-} mice. Groups of C57BL/6 WT or MR1^{-/-} mice (n=5-8/group) were injected i.v. with (A) 1×10⁵ B16F10 melanoma cells, (B) 5×10⁵ LWT1 melanoma cells, or (C, D) 5×10⁴ B16F10 melanoma cells on day 0. (E) Groups of bone marrow chimeric mice (n=8-10/group) were injected i.v. with 1×10⁵ B16F10 cells 10 weeks after bone marrow transplantation. In some groups, mice were treated i.p. with (C) cIg or anti-ASGM1 (50 μg/mouse, day −1, 0 and 7), (D) cIg or anti-IFNγ (750 μg/mouse, day −1; 250 μg/mouse, days 0, 7) relative to tumor cell inoculation. On day 14 relative to tumor cell inoculation, lungs were harvested and the metastatic burden was quantified by counting colonies on the lung surface. Data presented as mean±SEM. (F, G) Groups of C57BL/6 WT and MR1^{-/-} mice (n=21/group) were injected s.c. with MCA at (F) 25 μg or (G) 300 μg. Mice were subsequently monitored for tumor development over 250 days. Kaplan-Meier curves for overall survival of each group are shown. (H-J) Groups of C57BL/6 WT mice and MR1^{-/-} mice (n=6-8/group) were injected s.c. with 1×10⁶ SM1WT1 melanoma cells on day 0. In some groups, mice were either treated i.p. with (I) cIg or anti-ASGM1 (50 μg/mouse), (J) cIg (250 μg/mouse) or anti-CD8β (100 μg/mouse), or anti-IFNγ (250 μg/mouse) on days −1, 0, 7 and 14 relative to tumor cell inoculation. Mice were monitored for tumor growth (calculated by the product of 2 perpendicular axes). The data shows the mean tumor size (mm²)±SEM. In (A), the experiment was performed using WT and MR1^{-/-} mice that were co-housed in the same cages or separate cages while in (B-J) WT and MR1^{-/-} mice were all co-housed. Experiments were performed once for (A, C-J) while (B) is pooled from 3 independent experiments. Significant differences between groups as indicated by crossbars were determined using a one-way ANOVA followed by Tukey's post-hoc test (A, C-E, I-J), a Mann-Whitney test (B, H), or a log-rank (Mantel-Cox) test for (F, G), *p<0.05, **p<0.01, ***p<0.001, ****p<0.0001.

FIG. 2. Up-regulation of MR1 on B16F10 cells increases lung metastases that is dependent on MAIT cells. (A) The schematic and timeline of expanding MAIT cells from splenocytes derived from C57BL/6 WT mice with IL-2 and 5-OP-RU, for sorting and adoptive transfer into tumor-bearing mice. (B, C) Groups of WT or MR1^{-/-} or Rag2cγ^{-/-} mice (n=5-6/group) were injected i.v. with (B) 1×10⁵ or (C) 1×10⁴ B16F10 melanoma cells. In some groups, sorted MAIT or T conventional cells (_{c}T) (non-MAIT αβ⁺ T cells) (2×10⁵ cells/mouse) from C57BL/6 (B) WT or (C) TCRδ^{-/-} mice were i.v. injected into the indicated groups of mice one day before tumor inoculation. (C) One group of mice received i.v injection of media alone as a control. (B, C) On day 14 relative to tumor cell inoculation, lungs were harvested and the metastatic burden was quantified by counting colonies on the lung surface. Data presented as mean±SEM. (D) B16F10 or LWT1 melanoma cells were stimulated in vitro with DMSO or 5-OP-RU (100 nM) for 4 hours before cell surface MR1 expression was determined by flow cytometry. Groups of C57BL/6 WT or MR1^{-/-} mice (n=5-6/group) were i.v. injected with (E) 1×10⁵ B16F10 or (F) 5×10⁵ LWT1 cells melanoma cells pulsed with DMSO or 5-OP-RU (100 nM, 4 hours). Fourteen days later relative to tumor cell inoculation, lungs were harvested and the metastatic burden was quantified by counting colonies on the lung surface. Data presented as mean±SEM. Experiments performed once for (B, C, F) and three times for (D, E) with one representative experiment shown. Significant differences between groups as indicated by crossbars were determined using a one-way ANOVA followed by Tukey's post-hoc test (B, E, F), *p<0.05, **p<0.01, ****p<0.0001

FIG. 3. Upregulation of surface MR1 on tumor cells suppresses NK cell function through MAIT cells. (A) Schematic to analyze NK effector function in the lungs of C57BL/6 WT or MR1^{-/-} mice injected with 5-OP-RU-pulsed B16F10 or LWT1 cells. On day 5 relative to tumor inoculation, lungs were harvested from the indicated groups of mice (n=5-6/group) and stimulated with PMA/ionomycin plus protein transport inhibitors and NK cell effector function was assessed by flow cytometry. (B) Representative contour plots of IFNγ staining in NK cells (NKp46⁺ NK1.1⁺ TCRβ⁻ CD45.2⁺) and (C) the proportion and MFI of IFNγ⁺ NK cells amongst total NK cells in B16F10-bearing lungs. (D) Representative contour plots of CD107a staining in NK cells and (E) the proportion and MFI of CD107a⁺ NK cells amongst total NK cells in B16F10 tumor-bearing lungs. (F) Representative contour plots of IFNγ staining in NK cells and (G) the proportion and MFI of IFNγ⁺ NK cells amongst total NK cells in LWT1 tumor-bearing lungs. (H) Representative contour plots of CD107a staining in NK cells and (I) the proportion and MFI of CD107a⁺ NK cells amongst total NK cells in the LWT1-bearing lungs. Data presented as mean±SEM. Significant differences between groups as indicated by crossbars were determined using a one-way ANOVA followed by Tukey's post-hoc test, *p<0.05, **p<0.01, ***p<0.001, ****p<0.0001.

FIG. 4. Upregulation of MR1 on B16F10 activates MAIT cells to suppress NK cell effector function through IL-17. (A) Schematic to analyze MAIT or NK cell effector function in the lungs of C57BL/6 WT or MR1^{-/-} mice injected with 5-OP-RU- or DMSO-stimulated B16F10 melanoma cells. On day 5 relative to tumor inoculation, lungs were harvested and stimulated with PMA/ionomycin plus protein transport inhibitors for 4 hours and MAIT or NK cell function was assessed by flow cytometry. (B) The proportion of CD69⁺ MAIT cells amongst total MAIT cells (B220⁻ F4/80⁻ MR1-5-OP-RU tetramer⁺ TCRβ⁺) in naïve or tumor-bearing lungs. Data presented as mean±SEM. (C) Representative dot plot of IL-17 expression by MAIT cells. The (D) proportion of IL-17⁺ MAIT cells amongst total MAIT cells in the lungs of naïve and tumor-bearing mice as indicated. Data presented as mean±SEM. (E) Representative dot plot of TNF expression by MAIT cells. The (F) proportion of TNF MAIT cells and (G) IFNγ⁺ MAIT cells amongst total MAIT cells in the lungs of naïve and tumor-bearing mice as indicated. (H, I) On day 5 relative to tumor inoculation, lungs were harvested (n=6/group) and stimulated with PMA/ionomycin plus protein transport inhibitors for 3 hours and NK cell function was assessed by flow cytometry. The proportion of IFNγ⁺ NK cells and CD107a⁺ NK cells amongst total NK cells (NKp46⁺ NK1.1⁺ TCRβ⁻ CD45.2⁺) in tumor-bearing C57BL/6 WT or IL-17^{-/-} mice. Data presented as mean±SEM. (J) Groups of WT or MR1^{-/-} mice (n=4-6/group) were injected i.v. with 1×10⁵ B16F10 melanoma cells. In some groups, sorted MAIT cells (2×10⁵ cells/mouse) from C57BL/6 WT or IL-17A^{-/-} mice were i.v. injected into the indicated groups of mice one day before tumor inoculation. On day 14 relative to tumor cell inoculation, lungs were harvested and the metastatic burden was quantified by counting colonies on the lung surface. Data presented as mean±SEM. Data pooled from two independent experiments for (B, D, F, G). Experiments were performed once for (H-K). Significant differences between groups as indicated by crossbars were determined using a one-way ANOVA followed by Tukey's post-hoc test (B, D, F, J), or a Mann-Whitney test (H, I), *p<0.05, **p<0.01, ***p<0.001, ****p<0.0001.

FIG. 5. Expression of MR1 on tumor cells is critical for the suppressive function of MAIT cells. (A) Generation of three independent B16F10 cell lines knocked out for MR1 using three different MR1 sgRNA. Loss of MR1 surface expression on these cells with or without 5-OP-RU ligand stimulation was verified by flow cytometry. Empty vector transfected B16F10 cells were used as a positive control. (B, C) Groups of C57BL/6 WT, MR1^{-/-} or Rag2cγ^{-/-} mice (n=5-6/group) were i.v. injected with either (B) 1×10⁵ B16F10 vector control cells or the indicated clone of B16F10 cells lacking MR1 (sgR1, sgR2, sgR3) or (C) 1×10⁴ B16F10-MR1^{KO} cells (sgR3 clone). On day 14 relative to tumor cell inoculation, lungs were harvested and the metastatic burden was quantified by counting colonies on the lung surface. Data presented as mean±SEM. (D) Re-expression of GFP or GFP-MR1 expressing vector into B16F10-MR1^{KO} (sgR3 clone). MR1 surface expressions of these cells stimulated with or without 5-OP-RU were assessed by flow cytometry. (E) Groups of C57BL/6 WT mice (n=6/group) were i.v. injected with 1×10⁵ B16F10-MR1^{KO} (sgR3 clone) transfected with empty GFP-expressing vector or MR1-GFP-expressing vector. (F) Groups of C57BL/6 WT or MR1^{-/-} mice were i.v. injected with parental B16F10 cells treated with vehicle (dd H₂O) or AC-6-FP (10 μM, 18 hours). On day 14 relative to tumor cell injection, lungs were harvested and the metastatic burden was quantified by counting colonies on the lung surface. Data presented as mean±SEM. Experiments performed twice for (A-E) and once for (F). Significant differences between groups as indicated by crossbars were determined by a one-way ANOVA followed by Tukey's post-hoc test (A, F), or a Mann-Whitney test (C, E), *p<0.05, **p<0.01, ****p<0.0001.

FIG. 6. Anti-MR1 blocking antibodies suppress B16F10 and LWT1 experimental lung metastases. Groups of C57BL/6 WT or MR1^{-/-} mice (n=5-6/group) were i.v. injected with (A, B) 1×10⁵ B16F10, (C) 1×10⁵ parental B16F10 or 2×10⁵ B16F10-MR1^{KO} (sgR3 clone) melanoma cells and (D) 5×10⁵ LWT1 melanoma cells or s.c injected with (E) 1×10⁶ MCA1956 fibrosarcoma or (F) 1×10⁶ SM1WT1 melanoma cells on day 0. The indicated groups of mice were i.p. treated with cIg or anti-MR1 (clone 25.6 or clone 8F2.F9) (250 μg/mouse) at the indicated doses on days (A, B) −1, 0, 3 and 7, or (B, C, D) −1, 0 and 3 or (E, F) 6, 10, 14 and 18 relative to tumor cell inoculation. (A-D) On day 14, lungs were harvested and the metastatic burden was quantified by counting colonies on the lung surface. Data presented as mean±SEM. (E, F), Mice were monitored for tumor growth (calculated by the product of 2 perpendicular axes). The data shows the mean tumor size (mm²)±SEM. (G-K), From (F), end-stage tumors were harvested on day 26, and single cell suspensions stimulated with PMA/Ionomycin plus protein transport inhibitors for 4 hours. The proportions of (G) total CD45.2⁺ immune cells (H) CD8⁺ T cells (CD8⁺TCR⁺CD45.2⁺) and (I) NK cells (NK1.1⁺NKp46⁺TCRβ⁻CD45.2⁺) amongst CD45.2⁺ cells. The proportions of CD8⁺ T or NK cells that are (J-K) IFNγ⁺, (L-M) CD107a⁺ amongst CD8⁺ T or NK cells, respectively. All experiments performed once except (C) which was performed twice. Significant differences between groups as indicated by crossbars were determined by a one-way ANOVA followed by Tukey's post-hoc test (A, B) or a Mann-Whitney test (C, M), *p<0.05, **p<0.01, ***p<0.001.

FIG. 7. Anti-MR1 blocking antibodies suppress fibrosarcoma. Groups of C57BL/6 WT mice (n=20-21/group) were injected subcutaneously with 300 μg of MCA. Mice were treated with cIg or anti-MR1 (8F2.F9; 250 μg i.p., twice/week) for 6 weeks from the second palpable tumor measurement (0.19-0.38 cm², days 77-126 relative to MCA inoculation). Mice were monitored for fibrosarcoma development over 200 days, with measurements made with a caliper square as the product of two perpendicular diameters (cm2). Data recorded as tumor size in cm² of individual mice. Tumor growth following treatment was also determined by dividing the change in tumor size by the number of days after treatment initiation. Growth rate of each individual mouse is plotted as mean (mm²/day)±SEM.

FIG. 8. Anti-MR1 blocking antibodies suppress OS18 experimental osteosarcoma. Groups of C57B/L6 WT mice (n=9/group) were s.c. injected with 1×10⁶ OS18 osteosarcoma cells. Mice were treated with 250 μg cIg or anti-MR1 (clone 8F2.F9) on days 6, 10, 14 and 18 relative to tumour inoculation. Mice were monitored for tumor growth (calculated by the product of two perpendicular axes). (A) mean tumor size (mm²)±SEM (n=8/group), (B) individual growth curves (n=9/group).

FIG. 9. Anti-MR1 in combination with anti-PD1 suppress SM1WT1 experimental melanoma. Groups of C57BL/6 WT mice (n=7-9/group) were injected s.c. with 1×10⁶ SM1WT1 melanoma cell. Mice were i.p. treated with 250 μg cIg (1-1) or anti-MR1 (clone 8F2.F9) or anti-PD1 (RMP1-14) on days 6, 10, 14 and 18 relative to tumor inoculation. Mice were monitored for tumor growth (calculated by the product of two perpendicular axes). Experiment was performed once. The data shows the mean tumor size (mm2)±SEM. Significant differences between the indicated groups were determined using one-way ANOVA followed by Tukey post-hoc test, *p<0.05, ****p<0.0001.

FIG. 10. Immunohistochemical detection of MR1. Detection of MR1 in (A) melanoma and (B) colorectal carcinoma. Samples are scored on their tumor cell expression of MR1; SOX10+ (melanoma) and H&E (CRC) staining was used to verify tumor cells. All samples were stained using abcam polyclonal antibody 229715. Object lens 20×. A. Melanoma samples 0, 1+, 2+ and 3+ are a stage IV skin metastasis, stage III lymph node metastasis, stage IV lung metastasis and stage III brain metastasis, respectively. B. CRC samples are all stage IV; 0, 1+, 2+ and 3+ are a lymph node metastasis, primary tumor, visceral metastasis and primary tumor, respectively.

### KEY TO THE SEQUENCE LISTING

SEQ ID NO: 1 Amino acid sequence for a reference human MR1 (Q95460).
SEQ ID NO: 2 Nucleotide sequence of the heavy chain of antibody 26.5.
SEQ ID NO: 3 Nucleotide sequence of the light chain of antibody 26.5.
SEQ ID NO: 4 Amino acid sequence of the heavy chain of antibody 26.5.
SEQ ID NO: 5 Amino acid sequence of CDR1 of the heavy chain of antibody 26.5.
SEQ ID NO: 6 Amino acid sequence of CDR2 of the heavy chain of antibody 26.5.
SEQ ID NO: 7 Amino acid sequence of CDR3 of the heavy chain of antibody 26.5.
SEQ ID NO: 8 Amino acid sequence of the light chain of antibody 26.5.
SEQ ID NO: 9 Amino acid sequence of CDR1 of the light chain of antibody 26.5.
SEQ ID NO: 10 Amino acid sequence of CDR2 of the light chain of antibody 26.5.
SEQ ID NO: 11 Amino acid sequence of CDR3 of the light chain of antibody 26.5.
SEQ ID NO: 12 Nucleotide sequence of the heavy chain of antibody 8F2.F9.
SEQ ID NO: 13 Nucleotide sequence of the light chain of antibody 8F2.F9.
SEQ ID NO: 14 Amino acid sequence of the heavy chain of antibody 8F2.F9.
SEQ ID NO: 15 Amino acid sequence of CDR1 of the heavy chain of antibody 8F2.F9.
SEQ ID NO: 16 Amino acid sequence of CDR2 of the heavy chain of antibody 8F2.F9.
SEQ ID NO: 17 Amino acid sequence of CDR3 of the heavy chain of antibody 8F2.F9.
SEQ ID NO: 18 Amino acid sequence of the light chain of antibody 8F2.F9.
SEQ ID NO: 19 Amino acid sequence of CDR1 of the light chain of antibody 8F2.F9.
SEQ ID NO: 20 Amino acid sequence of CDR2 of the light chain of antibody 8F2.F9.
SEQ ID NO: 21 Amino acid sequence of CDR3 of the light chain of antibody 8F2.F9.

### DESCRIPTION OF EMBODIMENTS

The present disclosure is described in further detail with reference to one or more embodiments, some examples of which are illustrated in the accompanying figures. The examples and embodiments are provided by way of explanation and are not to be taken as limiting to the scope of the disclosure. Furthermore, features illustrated or described as part of one embodiment may be used by themselves to provide other embodiments and features illustrated or described as part of one embodiment may be used with one or more other embodiments to provide further embodiments. The present disclosure covers these variations and embodiments as well as other variations and/or modifications.

### General Techniques and Definitions

Unless specifically defined otherwise, technical and scientific terms used herein shall be taken to have the same meaning as commonly understood by one of ordinary skill in the art (e.g., in immunology, immunohistochemistry, protein chemistry, cell biology, biochemistry and chemistry).

Unless otherwise indicated, the recombinant protein, cell culture, and immunological techniques utilized in the present disclosure are standard procedures, known to those skilled in the art, such as those described in J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including updates until present).

The term “and/or”, e.g., “X and/or Y” shall be understood to mean either “X and Y” or “X or Y” and shall be taken to provide explicit support for both meanings or for either meaning.

### MR1 and MAIT Cells

Mucosal-associated invariant T (MAIT) cells represent a population of T cells that display a semi-invariant T cell receptor (TCR) and are restricted by the evolutionarily conserved major histocompatibility complex (MHC) class I-related protein I (MR1) (Tilloy et al., 1999; Le Bourhis et al., 2011). In mouse and humans, MAIT cells are developmentally and functionally dependent on MR1 and the host microbiota. Unlike classical MHC molecules, MR1 presents non-protein antigens. Studies have demonstrated that MR1 presents transient metabolites derived from the microbial synthesis of vitamin B2 and B9 to activate MAIT cells to rapidly secrete effector cytokines including interferon γ (IFNγ), tumor necrosis factor (TNF), and interleukin 17. These MR1-bound antigens can be stimulatory or non-stimulatory to MAIT cells. To date, 5-OP-RU, an intermediate of the riboflavin (vitamin B2) biosynthesis pathway, is one of the most potent stimulatory MAIT cell antigens. In contrast, folate (vitamin B9)-based MR1 ligands such as 6-formylpterin (6-FP) and its derivative acetyl 6-formylpterin (AC-6-FP) inhibit MAIT cell activation. It was also reported that other molecules including organic molecules, drugs, drug metabolites and drug-like molecules, including salicylates and diclofenac acted as MR1-binding ligands that inhibited or activated MAIT cells. In addition to activation through the interaction of MR1-TCR, MAIT cells can also be activated in an MR1-independent, but cytokine-dependent manner to produce effector cytokines.

MAIT cells have been proposed to have anti-tumor activity as, in vitro, it was demonstrated that they displayed cytolytic activity against tumor cells when cultured at high effector/target ratio in the presence of MAIT cell antigen or following PMA/ionomycin stimulation.

The present disclosure shows that MAIT cells display tumor-promoting function and that blocking MR1, or saturating it with an inhibitory ligand provides a new therapeutic strategy for the treatment of cancer. In addition, the level of MR1 expression in tumor cells may be used for prognostic and predictive methods to determine the likely course of disease and/or likelihood of response to treatment.

### Therapeutic Methods

The present disclosure provides methods for the treatment of cancer in a subject, the methods comprising administering to the subject a compound that inhibits MR1-mediated cell activation.

The present disclosure also provides the use of a compound that inhibits MR1-mediated cell activation in the manufacture of a medicament for the treatment of cancer in a subject.

The present disclosure also provides a compound that inhibits MR1-mediated cell activation for use in the treatment of cancer in a subject.

As would be understood in the art, in some embodiments, the compound may be one which binds to MR1. By binding to MR1, the compound may prevent MR1 signalling and activation of MAIT cells, via the MAIT cell TCR.

As used herein, the term “cancer” refers to a disease characterized by the rapid and uncontrolled growth of cells. Cancer cells can spread locally or through the bloodstream and lymphatic system to other parts of the body. Examples of various cancers include, but are not limited to, thoracic cancer, including non-small cell lung cancer and small cell lung cancer, thymoma, thymic carcinoma, thyroid cancer and mesothelioma; head and neck cancer including of the oropharynx, nasopharynx and hypopharynx; melanoma including cutaneous, uveal and acral; skin cancer including basal cell carcinoma, merkel cell carcinoma and squamous cell carcinoma; neurological cancer including glioma, astrocytoma, oligodendroglioma and rare brain tumours; germ cell cancers of any primary site; sarcoma including all sub-types of soft tissue and bone; hepatobiliary cancer including liver, cholangiocarcinoma and gall bladder cancer; upper gastrointestinal cancers including oesophageal, gastric, pancreas and small bowel; lower gastrointestinal cancers including colon, rectal and anal; breast Cancer; CNS cancer; gynaecological cancer including ovarian, primary peritoneal, endometrial and vulval; genitourinary cancer including testicular, penile, prostate, bladder and kidney; neuroendocrine and adrenal cancers including carcinoid; cancer of unknown primary; lymphoma including Hodgkin and non-Hodgkin lymphomas, T-cell and B-cell lymphomas of all sub-types; leukaemia including lymphoid and myeloid leukaemia of all sub-types and plasma cell neoplasms including multiple myeloma; fibrosarcoma and osteosarcoma. In one example, the cancer is lung cancer. In one example, the cancer is non-small-cell lung carcinoma. In another example, the cancer is small-cell lung carcinoma. In another example, the cancer is osteosarcoma. In another example, the cancer is fibrosarcoma. In another example, the cancer is colorectal carcinoma. As used herein, the terms “treating”, “treat”, or “treatment” include administering a compound or molecule described herein to reduce, prevent, or eliminate at least one symptom of a disease or condition.

As used herein, the terms “preventing”, “prevent”, or “prevention” include administering a therapeutically effective amount of a compound or molecule sufficient to stop or hinder the development of at least one symptom of a disease or condition.

As used herein, the term “subject” shall be taken to mean any animal including humans, for example a mammal. Exemplary subjects include but are not limited to humans, mice and rats. In one example, the subject is a human. In another example, the subject is a mouse.

“Administering” as used herein is to be construed broadly and includes administering a compound or molecule as described herein to a subject as well as providing a compound or molecule as described herein to a cell.

As used herein, the term “inhibit” shall be taken to mean hinder, reduce, restrain or prevent MAIT cell activity in a MAIT cell relative to a MAIT cell in a subject to whom the compound that inhibits MRI-mediated MAIT cell activation has not been administered.

Inhibition of the MR1-restricted MAIT cell activation may be partial or complete. For example, MAIT cell activity may be reduced by at least by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% relative to the activity of a MAIT cell in a subject to whom the compound has not been administered.

Methods of measuring MAIT cell activity or activation are known in the art. For example, the production of cytokines such as IFN-γ, TNF-α, and IL-17 may be measured. After TCR-mediated activation, MAIT cells can become activated, which leads to cytokine production, cytotoxic effector function (may be measured by GrzB, perforin expression), migration and proliferative expansion. Upregulation of CD69 may also be used to identify activated MAIT cells (Godfrey et al., 2019).

It will be understood by a person skilled in the art that MR1-restricted MAIT cell activation may be inhibited directly or by inhibiting upstream or downstream effectors of MR1-mediated MAIT cell activation. Accordingly, the inhibitor may be a direct inhibitor of MRI or an indirect inhibitor of MRI. The inhibitor may bind to MRI to inhibit its function by changing its conformation or by affecting its active site.

Thus, for example the compound may be one which binds to MR1. The compound may be a ligand. By binding to MR1, the compound may prevent MR1 signalling and activation of MAIT cells, via the TCR. In one example, the compound may bind to MR1 and may inhibit MR1-mediated MAIT cell activation. In another example, the compound may bind to MR1 and may inhibit MR1 signalling of MAIT cells. Thus, the compound may block the initial contact between MR1 and a MAIT cell. The compound may also reduce binding of MRI to a MAIT cell. As used herein, MR1 signalling will be understood to mean the activation a MAIT cell by MR1.

Alternatively, the inhibitor of MR1 may inhibit a binding partner of MR1 and thereby affect MR1 function and signalling. For example, it has been shown that the deletion of genes encoding key enzymes in the riboflavin pathway abolishes an otherwise productive MAIT cell response to Lactococcus lactis, Salmonella enterica serovar Typhimurium or Escherichia coli (Kjer-Nielsen, L. et al. Nature 491, 717-723 (2012); Corbett, A. J. et al. Nature 509, 361-365 (2014); Soudais, C. et al. J. Immunol. 194, 4641-4649 (2015)). Likewise, inhibition of the riboflavin operon in L. lactis also abolishes the activation of MAIT cells (Corbett, A. J. et al. Nature 509, 361-365 (2014)).

In some embodiments, the compounds as described herein may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, or other therapeutic agents.

According to at least some embodiments, the compound as described herein may be used in combination with any of the known in the art standard of care cancer treatments (as can be found, for example, on the World Wide Web at cancer.gov/cancertopics).

In some embodiments, conventional/classical anti-cancer agents suitable for use in the therapeutic methods described herein include but are not limited to platinum based compounds, antibiotics with anti-cancer activity, Anthracyclines, Anthracenediones, alkylating agents, antimetabolites, Antimitotic agents, Taxanes, Taxoids, microtubule inhibitors, Folate antagonists and/or folic acid analogs, Topoisomerase inhibitors, Aromatase inhibitors, GnRh analogs, inhibitors of 5α-reductase, bisphosphonates; pyrimidine analogs, purine analogs and related inhibitors, vinca alkaloids, epipodophyllotoxins, antibiotics, L-Asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog.

Specific but non-limiting examples of these categories of drugs are as follows: platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antiestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; inhibitors of 5.alpha.-reductase such as finasteride.

Therapeutic antibodies which may optionally be used in combination with the therapeutic methods described herein include but are not limited to cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept, pembrolizumab, atezolizumab, avelumab, durvalumab and nivolumab. In one example, the therapeutic antibody is an anti-PD1 antibody. The anti-PD1 antibody may be pembrolizumab, nivolumab or cemiplimab. In another example, the therapeutic antibody is an anti-PD-L1 antibody. The anti-PD-L1 antibody may be atezolizumab, avelumab or durvalumab.

### Compounds

A “compound” that binds to or inhibits MR1, as contemplated by the present disclosure, can take any of a variety of forms including natural compounds, chemical small molecule compounds or biological compounds or macromolecules. Exemplary compounds include an antibody or an antigen binding fragment of an antibody, a nucleic acid, a polypeptide, a peptide, a ligand, and a small molecule.

### Antibodies

In some embodiments, the compound that binds to MR1 and prevents or inhibits MR1-dependent activation of MAIT cells is an antibody. The skilled artisan will be aware that an “antibody” is generally considered to be a protein that comprises a variable region made up of a plurality of polypeptide chains, e.g., a polypeptide comprising a VL and a polypeptide comprising a VH. An antibody also generally comprises constant domains, some of which can be arranged into a constant region, which includes a constant fragment or fragment crystallizable (Fc), in the case of a heavy chain. A VH and a VL interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG1, IgG2, IgG3, IgG4, IgA1 and IgA2) or subclass. The term “antibody” also encompasses humanized antibodies, primatized antibodies, human antibodies and chimeric antibodies.

As used herein, the term “Fv” shall be taken to mean any protein, whether comprised of multiple polypeptides or a single polypeptide, in which a VL and a VH associate and form a complex having an antigen binding site, i.e., capable of specifically binding to an antigen. The VH and the VL which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

In some embodiments, the antibody may be a single domain antibody (sdAb), for example a nanobody. An sdAb is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

As used herein, the term “binds” in reference to the interaction of a compound or an antigen binding site thereof with an antigen means that the interaction is dependent upon the presence of a particular structure (e.g., an antigenic determinant or epitope) on the antigen.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a compound binds to MR1 with materially greater affinity (e.g., 20 fold or 40 fold or 60 fold or 80 fold to 100 fold or 150 fold or 200 fold) than it does to other receptors or to antigens commonly recognized by polyreactive natural antibodies (i.e., by naturally occurring antibodies known to bind a variety of antigens naturally found in humans).

In some embodiments, the antibody that binds MR1 may be a multi-specific antibody, such as a bi-specific antibody, e.g. composed of two different fragments of two different antibodies, such that the bi-specific antibody binds two types of antigen. For example, a bi-specific antibody may comprise a fragment that binds MR1, and a second fragment that binds to a second antigen. The second antigen may be, for example, a marker of an immune cell, for example such as CD56 on NK cells, or a marker specific for CD4⁺ T-cells or CD8⁺ T-cells. In one example, the second antigen may be PDL1. In another example, the second antigen may be a tumor targeting antigen. Techniques for the preparation of bi-specific antibodies are well known in the art.

As used herein, the term “antigen binding site” shall be taken to mean a structure formed by a protein that is capable of binding or specifically binding to an antigen. The antigen binding site need not be a series of contiguous amino acids, or even amino acids in a single polypeptide chain. For example, in a Fv produced from two different polypeptide chains the antigen binding site is made up of a series of amino acids of a VL and a VH that interact with the antigen and that are generally, however not always in the one or more of the CDRs in each variable region. In some examples, an antigen binding site is a VH or a VL or a Fv.

As used herein, the term “epitope” (syn. “antigenic determinant”) shall be understood to mean a region of an antigen, for example MR1, to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when MR1 is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The Fc region of an antibody interacts with a number of ligands (also referred herein as “Fc ligands” which include but are not limited to agents that specifically bind to the Fc region of antibodies, including Fc receptors and C1q, imparting functional capabilities referred to as effector functions. The Fc receptors mediate communication between antibodies and the cellular arm of the immune system. In humans this protein family includes FcγRI (CD64), including isoforms FcγRIA, FcγRIB, and FcγRIC; FcγRII (CD32), including isoforms FcγRIIA, FcγRIIB, and FcγRIIC; and FcγRIII (CD16), including isoforms FcγRIIIA and FcγRIIB. These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). In addition, an overlapping site on the Fc region of the molecule also controls the activation of a cell independent cytotoxic function mediated by complement, otherwise known as complement dependent cytotoxicity (CDC).

In some circumstances, the binding and stimulation of effector functions mediated by the Fc region of immunoglobulins may be beneficial, however, in certain instances it may be more advantageous to decrease or eliminate effector function. For example, where blocking the interaction of a receptor with its cognate ligand is the objective, it may be advantageous to decrease or eliminate other antibody effector function. Accordingly, in some embodiments, the antibodies used in the methods described herein may have reduced or ablated Fc receptor function. Methods for producing Fc modified antibodies are known in the art, and include methods that use the addition, substitution, or deletion of one or more amino acid residues in the Fc region, or alternatively the modification of Fc glycosylation. A “variant Fc region” or “engineered Fc region” comprises an amino acid sequence that differs from that of a native-sequence Fc region by virtue of at least one amino acid modification, preferably one or more amino acid substitution(s). In some embodiments, the variant Fc region has at least one amino acid substitution compared to a native-sequence Fc region or to the Fc region of a parent polypeptide, e.g., from about one to about ten amino acid substitutions, or from about one to about five amino acid substitutions in a native-sequence Fc region or in the Fc region of the parent polypeptide. In some embodiments, Variant Fc sequences for a “dead Fc” may include three amino acid substitutions in the CH2 region to reduce FcγRI binding at EU index positions 234, 235, and 237 (see, e.g., Duncan et al., 1988). Two amino acid substitutions in the complement C1q binding site at EU index positions 330 and 331 reduce complement fixation. In addition, substitution into human IgG1 of IgG2 residues at positions 233-236 and IgG4 residues at positions 327, 330 and 331 greatly reduces ADCC and CDC (see, for example, Armour et al., 1999).

As known in the art, antibodies that bind to MR1 are commercially available, for example such as monoclonal anti-MR1 antibodies supplied by Sigma-Aldrich (product #WH0003140M3 and SAB1403923). Accordingly, in some embodiments, the antibody may be derived from a commercially available antibody or may comprise an antigen binding fragment from a commercially available antibody. In some embodiments, the antibody may be antibody 26.5 as described herein. In some embodiments, the heavy chain of the antibody may be encoded by the sequence set forth in SEQ ID NO: 2 and the light chain of the antibody may be encoded by the sequence set forth in SEQ ID NO: 3. In some embodiments, the heavy chain of the antibody may have the sequence set forth in SEQ ID NO: 4 and the light chain may have the sequence set forth in SEQ ID NO: 8. In some embodiments, the antibody comprises CDRs having amino acid sequences that are at least 75%, or at least 80%, or at least 85%, or at least 90%, or at least 95%, or at least 96%, or at least 97%, or at least 98%, or at least 99%, or at least 99.5%, or at least 99.9% identical to the amino acid sequences set forth in SEQ ID NOs: 5-7 and 9-11. In some embodiments, the antibody comprises CDRs having the amino acid sequences set forth in SEQ ID NOs: 5-7 and 9-11. In some embodiments, the antibody may be antibody 8F2.F9, as described herein. In some embodiments, the heavy chain of the antibody may be encoded by the sequence set forth in SEQ ID NO: 12 and the light chain of the antibody may be encoded by the sequence set forth in SEQ ID NO: 13. In some embodiments, the heavy chain of the antibody may have the sequence set forth in SEQ ID NO: 14 and the light chain may have the sequence set forth in SEQ ID NO: 18. In some embodiments, the antibody comprises CDRs having amino acid sequences that are at least 75%, or at least 80%, or at least 85%, or at least 90%, or at least 95%, or at least 96%, or at least 97%, or at least 98%, or at least 99%, or at least 99.5%, or at least 99.9% identical to the amino acid sequences set forth in SEQ ID NOs: 15-17 and 19-21. In some embodiments, the antibody comprises CDRs having the amino acid sequences set forth in SEQ ID NOs: 15-17 and 19-21. In some embodiments, the antibodies may be bi-specific T-cell engager antibodies (also referred to as BiTes). As known in the art, bi-specific T-cell engager antibodies are a class of artificial bispecific monoclonal antibodies that direct a host's immune system, more specifically cytotoxic T cell activity, against cancer cells. BiTes are fusion proteins consisting of two single-chain variable fragments (scFvs) of different antibodies on a single peptide chain. One of the scFVs binds to T cells and the other bind to a tumor molecule via a tumor specific molecule. Due to the link created between T cells and tumor cells, the T cells are able to exert cytotoxic activity on tumor cells through production of proteins such as perforin and granzymes in order to initiate tumor cell apoptosis. Thus, in some embodiments, the antibody may be a bi-specific T-cell engager that binds to T cells and MR1 on tumor cells.

### Protein/Peptide Inhibitors

In some embodiments, the compound that binds to and/or blocks MR1 is a protein or peptide. The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions. The term “polypeptide” or “polypeptide chain” will be understood from the foregoing paragraph to mean a series of contiguous amino acids linked by peptide bonds.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

### Nucleic Acid Inhibitors

In some embodiments, the therapeutic and/or preventative methods as described herein involve reducing expression of MR1. For example, such a method may involve administering a compound that reduces transcription and/or translation of a nucleic acid encoding MR1. In one example, the compound that inhibits MR1 activity is a nucleic acid, e.g., an antisense polynucleotide, a ribozyme, a PNA, an interfering RNA, a siRNA, or a microRNA.

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding MR1. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure, such as a short hairpin RNA (shRNA). The design and production of suitable dsRNA molecules for the present disclosure is well within the capacity of a person skilled in the art, particularly considering WO99/32619, WO99/53050, WO99/49029, and WO01/34815. Such dsRNA molecules for RNAi include, but are not limited to short hairpin RNA (shRNA) and bi-functional shRNA.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search.

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding MR1, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding MR1. The full-length sequence complementary to the entire gene transcript may be used. The degree of identity of the antisense sequence to the targeted transcript should be at least 90%, for example, 95-100%.

In another example, the compound may be a nucleic acid aptamer (adaptable oligomer). Aptamers are single stranded oligonucleotides or oligonucleotide analogs that are capable of forming a secondary and/or tertiary structure that provides the ability to bind to a particular target molecule, such as a protein or a small molecule, e.g., MR1. Thus, aptamers are considered the oligonucleotide analogy to antibodies. In general, aptamers comprise about 15 to about 100 nucleotides, such as about 15 to about 40 nucleotides, for example about 20 to about 40 nucleotides, since oligonucleotides of a length that falls within these ranges can be prepared by conventional techniques.

An aptamer can be isolated from or identified from a library of aptamers. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., binds specifically to MR1) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990; U.S. Pat. Nos. 5,270,163; and/or 5,475,096.

### MR1 Ligands

In an embodiment, the present invention provides ligands or small molecule inhibitors useful for modulating MR1 binding and/or signalling of MAIT cell TCRs, thus reducing MAIT cell activation.

The semi-invariant and evolutionary conserved nature of the MAIT TCR indicates that MAIT cells are specific for a limited class of antigens presented by MR1. The ligand-binding groove of MR1 is ideally suited to present small organic compounds that can originate from vitamins (microbial-derived metabolites) rather than antigenic peptides. MR1 bound ligands can be stimulatory or inhibitory to MAIT cells. Folate (vitamin B9)-based MR1 ligands such as 6-formyl pterin (6-FP) and its derivative Acetyl 6-formyl pterin (Ac-6-FP) inhibit MAIT cell activation.

The ligands or compounds may be prepared in a variety of ways. Conveniently, they can be synthesized by conventional techniques employing automatic synthesizers, or may be synthesized manually. The ligands may also be isolated from natural sources and purified by known techniques, including, for example, chromatography on ion exchange materials, separation by size, immunoaffinity chromatography and electrophoresis.

### Cancer Immunotherapies

In some embodiments, a compound as described herein is administered to a patient in combination with one or more cancer immunotherapies, including cell-based therapies, CAR-T cell therapies, antibody therapies, cytokine therapies, and other immunosuppressive mediators such as indoleamine 2,3-dioxygenase (IDO) or transforming growth factor-β (TGF-β). Unless otherwise indicated, “in combination” as used herein includes substantially simultaneous administration of the compound and one or more cancer immunotherapies (either in the same composition or in separate compositions) as well as sequential administration.

Cell-based therapies include, but are not limited to, CAR-T cell therapy, natural killer cells, lymphokine-activated killer cells, cytotoxic T cells, regulatory T cells, and dendritic cells. Cytokine therapies include, but are not limited to, GM-CSF, interleukins (e.g., IL-2, IL-7, IL-10, IL-12, IL-15, IL-18, IL-21), and interferons (e.g., interferon alpha).

In some embodiments, the methods of treatment disclosed herein may be used in combination or in conjunction with other cancer immunotherapies and immunotherapy agents.

Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines.

Among these, multiple antibody therapies are approved in various jurisdictions to treat a wide range of cancers. Cell surface receptors are common targets for antibody therapies and include CD20, CD274 and CD279. Once bound to a cancer antigen, antibodies can induce antibody-dependent cell-mediated cytotoxicity, activate the complement system, or prevent a receptor from interacting with its ligand, all of which can lead to cell death. Approved antibodies include alemtuzumab, ipilimumab, nivolumab, ofatumumab and rituximab.

Active cellular therapies usually involve the removal of immune cells from the blood or from a tumor. Those specific for the tumor are cultured and returned to the patient where they attack the tumor; alternatively, immune cells can be genetically engineered to express a tumor-specific receptor, cultured and returned to the patient. Cell types that can be used in this way are natural killer cells, lymphokine-activated killer cells, cytotoxic T cells and dendritic cells. One specific type of cellular therapy is CAR-T therapy. Chimeric antigen receptors (CARs, also known as chimeric immunoreceptors, chimeric T cell receptors or artificial T cell receptors) are engineered receptors which graft an arbitrary specificity onto an immune effector cell. Typically, these receptors are used to graft the specificity of a monoclonal antibody onto a T cell, with transfer of their coding sequence facilitated by retroviral vectors. The receptors are called chimeric because they are composed of parts from different sources. The basic principle of CAR T-Cell design involves recombinant receptors that combine antigen-binding and T-Cell activating functions. The general premise of CAR T-Cells is to rapidly generate T-Cells targeted to specific tumour cells. Scientists can remove T-cells from a patient, genetically engineer them, and then put them back into the patient to target cancer cells.

In some embodiments, the cancer immunotherapy agent may be selected from one or more of an immune checkpoint modulatory agent, a cancer vaccine, an oncolytic virus, a cytokine, and a cell-based immunotherapies.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

The PD-1/PD-L1 modulatory agent may be selected from the group consisting of pembrolizumab, nivolumab, cemiplimab, atezolizumab, avelumab and durvalumab. In one example, PD-1 modulatory agent may be pembrolizumab, nivolumab or cemiplimab. In another example, PD-L1 modulatory agent may be atezolizumab, avelumab or durvalumab.

It will be appreciated that cancer immunotherapy methods involving the use of compounds inhibit MR1 activation of MAIT cells may be performed in isolation or as an adjunct to other known cancer therapy regimes. For example, treatment may be conducted in conjunction with or after treatments such as chemotherapy, radiation therapy, stem cell transplant and/or immunotherapy, for example, monoclonal antibody therapy. Examples of chemotherapeutic agents used in the treatment of brain tumors include temozolomide, BCNU (Carmustine), PCV (combination of procarbazine, CCNV (Lomustine), and vincristine), carboplatin, etoposide, irinotecan, Cis-Retonoic acid, thalidomide, tamoxifen and COX-2 inhibitors. Other known chemotherapeutic agents include chlorambucil, cyclophosphamide, melphalan, daunorubicin, doxorubicin, idarubicin, mitoxantrone, methotrexate, fludarabine, cytarabine, etoposide, topotecan, prednisone, dexamethasone, vincristine and vinblastine.

Identifying Candidate Compounds that Modulate MR1 Activity

In light of the present disclosure, the person skilled in the art may conduct screening assays to identify antibodies and molecules, including small molecules and ligands, that bind to and/or modulate the activity of MR1, such as inhibition of MAIT-cell activation, including the assays described herein. For example, the skilled person can perform MAIT cell activation assays in the presence of a test compound according to the methods described herein.

The activity of MAIT cells can be assessed by standard methods known in the art for assessing cellular activity. In a non-limiting example, the activity of MAIT cells is assessed in a assay in which MAIT cells are incubated in the presence or absence of MR1 expressing cells and test compounds, and with NK cells or T cells. The effect of the presence of the test compounds on the properties of the NK cells or T cells, for example, their proliferation, activity, cytotoxicity, or production of cytokines are assessed. In other examples, assays may examine MAIT cell surface activation markers such as CD69, IL2R by flow cytometric techniques; alternatively, cytokine production of MAIT cells in response to ligand stimulation or blockade might evaluate a broad array of cytokines production by flow-based, cytokine array methods. In a non-limiting example, MAIT cell activation can be assayed by CD69 upregulation in the case of MAIT TCR transduced cell lines or MAIT cells within PBMCs and intracellular cytokine staining for interferon (IFN) and tumor necrosis factor (TNF) in the case of MAIT cells derived from PBMCs.

The activity of MAIT cells can also be assessed by exposing the cells to the test compounds themselves and assessing its effect on any aspect of the cells' activity or behaviour. In such assays, a baseline level of activity (e.g., cytokine production, proliferation) of the MAIT cells is obtained in the absence of a ligand, and the ability of test compound to alter the baseline activity level is detected. In one such embodiment, a high-throughput screening approach is used to identify MR1 ligands capable of affecting the activation of the MAIT cell receptor. Examples of assays that can be used to assess MAIT cell activity can be found, inter alia, in U.S. Patent Application No. 20030215808; Kawachi et al. (2006), Huang et al., (2005), Treiner et al. (2005), Treiner et al. (2003).

Thus, the present disclosure provides methods to identify candidate compounds that block or inhibit MR1 activation of MAIT cells when administered to a cell, tissue, or subject. Such methods may be carried out in vivo, for example in animal subjects; or using in vitro and/or ex vivo assays, such as described herein.

### Compositions

Compositions comprising a compound that modulates MR1 signalling or activity together with an acceptable carrier or diluent are useful in the methods disclosed herein. Therapeutic compositions can be prepared by mixing the desired compounds having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances.

Therapeutic compositions to be used for in vivo administration should be sterile. This is readily accomplished by filtration through sterile filtration membranes, prior to or following lyophilization and reconstitution. The composition may be stored in lyophilized form or in solution if administered systemically. If in lyophilized form, it is typically formulated in combination with other ingredients for reconstitution with an appropriate diluent at the time for use. An example of a liquid formulation is a sterile, clear, colourless unpreserved solution filled in a single-dose vial for subcutaneous injection.

Single or multiple administrations of the compositions are administered depending on the dosage and frequency as required and tolerated by the patient. The dosage and frequency will typically vary according to factors specific for each patient depending on the specific therapeutic or prophylactic agents administered, the severity and type of disease or condition, the route of administration, as well as age, body weight, response, and the past medical history of the patient. Suitable regimens can be selected by one skilled in the art by considering such factors and by following, for example, dosages reported in the literature and recommended in the Physician's Desk Reference (56th ed., 2002). Generally, the dose is sufficient to treat or ameliorate symptoms or signs of disease without producing unacceptable toxicity to the patient.

### Predictive Methods

There is further provided methods for determining a likelihood of a positive or negative clinical response in a patient to a cancer therapy, the method comprising comparing the level of expression and/or activity of MR1 on tumor cells in the subject to a reference level of expression of MR1 on tumor cells,

wherein a higher level of expression and/or activity of MR1 on tumor cells in the subject compared to the reference level of expression and/or activity of MR1 on tumor cells is indicative of the patient having an increased likelihood of a positive clinical response to the cancer therapy, wherein the cancer therapy comprises administration of compound that inhibits MR1-mediated MAIT cell activation and/or that binds to MR1 on tumor cells in the patient.

The presence or level of expression and/or activity of MR1 may be determined by any method known in the art. Any of the methods disclosed herein to determine the presence or level of expression and/or activity of MR1 may used.

Any of the methods disclosed herein may comprise a step of establishing a reference level of MR1 expression and/or activity or a reference amount and/or activity of MAIT cells. Alternatively, any of the methods disclosed herein may comprise a step of comparing a measurement of MR1 expression and/or activity or amount and/or activity of MAIT cells to a predetermined reference level. Suitable threshold levels can then be determined according to the particular methodology used to identify and/or measure MR1 expression and/or activity and/or measure the amount and/or activity of MAIT cells. It will be appreciated that the precise thresholds will vary depending on the samples used to establish those threshold levels and according to the particular analytical methodology used in each instance. Thus, a “higher” amount and/or activity of MAIT cells is an amount and/or activity of MAIT cells that is increased relative to the reference amount and/or activity of MAIT cells. Conversely, a “normal” amount and/or activity of MAIT cells is an amount and/or activity of MAIT cells that is similar to, equal to, or greater than the reference amount and/or activity of MAIT cells. The “normal” amount and/or activity of MAIT cells or the reference amount and/or activity of MAIT cells can be determined by selecting any suitable population of cells from which to derive the amount and/or activity of MAIT cells.

Any of the methods disclosed herein may comprise a step of establishing a reference level of MR1 expression and/or activity. Alternatively, any of the methods disclosed herein may comprise a step of comparing a measurement of MR1 expression and/or activity to a predetermined reference level. Suitable threshold levels can then be determined according to the particular methodology used to identify and/or measure MR1 expression and/or activity. It will be appreciated that the precise thresholds will vary depending on the samples used to establish those threshold levels and according to the particular analytical methodology used in each instance. Thus, a “higher” level of MR1 expression and/or activity is a level of MR1 expression and/or activity that is increased relative to the reference level of MR1 expression and/or activity. Conversely, a “normal” level of MR1 expression and/or activity is a level of MR1 expression and/or activity that is similar to, equal to, or greater than the reference level of MR1 expression and/or activity. The “normal” level of MR1 expression and/or activity or the reference level of MR1 expression and/or activity can be determined by selecting any suitable population of cells from which to derive the level of MR1 expression and/or activity.

The methods described herein may also be used to determine the likelihood of cancer metastasis in a subject.

The terms ‘responsive”, “clinical response”, “positive clinical response” and the like, as used in the context of a patient's response to a cancer therapy, are used interchangeably and include reference to a favourable patient response to a drug as opposed to unfavourable responses, i.e. adverse events. In a patient, beneficial response can be expressed in terms of a number of clinical parameters, including loss of detectable tumor (complete response, CR), decrease in tumor size and/or cancer cell number (partial response, PR), tumor growth arrest (stable disease, SD), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis may be indicative of lack of beneficial response to treatment. In a population the clinical benefit of a drug, i.e., its efficacy can be evaluated on the basis of one or more endpoints. For example, analysis of overall response rate (ORR) classifies as responders those patients who experience CR or PR after treatment with drug. Analysis of disease control (DC) classifies as responders those patients who experience CR, PR or SD after treatment with drug.

A positive clinical response can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition of metastasis; (6) enhancement of anti-tumor immune response, possibly resulting in regression or rejection of the tumor; (7) relief, to some extent, of one or more symptoms associated with the tumor; (8) increase in the length of survival following treatment; and/or (9) decreased mortality at a given point of time following treatment. Positive clinical response may also be expressed in terms of various measures of clinical outcome. Positive clinical outcome can also be considered in the context of an individual's outcome relative to an outcome of a population of patients having a comparable clinical diagnosis, and can be assessed using various endpoints such as an increase in the duration of recurrence-free interval (RFI), an increase in the time of survival as compared to overall survival (OS) in a population, an increase in the time of disease-free survival (DFS), an increase in the duration of distant recurrence-free interval (DRFI), and the like. An increase in the likelihood of positive clinical response corresponds to a decrease in the likelihood of cancer recurrence.

### EXAMPLES

### Example 1. Materials and Methods

### Mice

C57BL/6 wild type (WT) and gene-targeted mice were bred in-house. C57BL/6 MR1^{-/-} mice were kindly provided by James McCluskey, Melbourne University, Australia. C57BL/6 IL-17A^{-/-} mice were provided by Geoffrey R. Hill, QIMR Berghofer Medical Research Institute, Australia. TCRδ^{-/-} mice were provided by Ian Frazer, The University of Queensland, Australia. Ragc2γ^{-/-} mice were generated at QIMR Berghofer Medical Research Institute by crossing Rag2^{-/-} mice with IL-2Rγ^{-/-} mice. WT and MR1^{-/-} mice were co-housed in the same cage for at least 4 weeks before experiment initiation unless specifically indicated. Age-matched mice were used in all experiments. All WT and gene-targeted mice were used between the ages of 6 to 14 weeks. All experiments were approved by the QIMR Berghofer Medical Research Institute Animal Ethics Committee.

### Cell Culture

Mouse melanoma cell lines B16F10, LWT1, SM1WT1 and MCA1956 were maintained as previously described (Yan et al., 2018; Blake et al., 2016; Ferrari de Andrade et al., 2014; Mittal et al., 2019). Mouse osteosarcoma cell lines OS18 were maintained as previously described (Kansara et al., 2019). Anti-MR1 antibody-producing hybridoma cell lines (clone 26.5 IgG2a isotype and 8F2.F9 IgG1 isotype) were maintained in complete RPMI 1640 media containing 10% fetal bovine serum, 1% L-glutamine and 1% penicillin/streptomycin. All cell lines were routinely tested for Mycoplasma.

### Antibodies and Reagents for In Vivo Experiments

Anti-CD8β (clone 53.5.8), anti-IFNγ (clone H22) and control IgG antibody (clone 1-1) were purchased from BioXcell (West Lebanon, N.H., USA). Anti-asialoGM1 (ASGM1) was purchased from Wako Pure Chemicals, Japan. The dose and schedule of antibody treatment were indicated in the Figure Legends. Anti-MR1 mAbs were purified from the supernatant of hybridoma cells by Protein G affinity resin column. Biotinylated MR1-antigen monomer was conjugated with PE-streptavidin (BioLegend, catalog #5544061) to generate MR1 tetramers. MAIT cell antigens 5-OP-RU and AC-6-FP was generated as described in Corbett et al., 2014. Cell stimulation cocktail (containing protein transport inhibitors) was purchased from Invitrogen Themofisher Scientific.

### Tumor Models

The indicated numbers of B16F10 and LWT1 cells were injected intravenously (i.v.) into the tail vein of WT or gene-targeted mice. In some experiments, B16F10 and LWT1 cells were treated with 5-OP-RU (100 nM, 4 h) or AC-6-FP (10 μM, 18 h), or their respective DMSO or ddH₂O vehicle controls before injection. Cells with viability greater than 90% were used in the experiments. Lungs were harvested on day 14, and surface tumor nodules were counted under a dissection microscope. For therapy experiments, anti-MR1 or cIg (clone 1-1, Leinco) were injected intraperitoneally into mice at the dose and schedule indicated in the figure legends. SM1WT1 or MCA1956 (both 1×10⁶) tumor cells or 1×10⁶ 0 S18 were injected subcutaneously into WT or gene-targeted male or female mice, respectively, prior to treatment with cIg or anti-MR1 at time points indicated in the figure legends. For MCA-induced fibrosarcoma, WT and MR1^{-/-} mice were injected subcutaneously (s.c.) in the hind flank with MCA (Sigma-Aldrich) in 100 μl of corn oil with the doses as indicated in the figure legends. For established tumor MCA experiments, mice were treated intraperitoneally with cIg or anti-MR1 from the second palpable tumor measurement twice a week for 6 weeks as indicated in the figure legend. Mice were monitored for fibrosarcoma development over 250 days. Tumor sizes were determined by caliper square measurements of two perpendicular diameters with data represented as mean±SEM (mm²) for each group.

### Bone Marrow Transplantation and Reconstitution

Bone marrow cells were obtained from the femurs of donor C57BL/6 WT (PTPRCA, CD45.1⁺) mice and MR1^{-/-} (C57BL/6, CD45.2⁺) mice. Two doses of 5.5 Gy of whole-body irradiation were administered to recipient WT and MR1^{-/-} mice at 4 hours interval. Recipient mice were injected i.v. with 5×10⁶ bone marrow cells/mouse after irradiation. Mice were provided with water containing neomycin for 4 weeks. Ten weeks after bone marrow transplantation, mice were eye-bled and immune cells were analyzed by flow cytometry using congenic CD45.1 and CD45.2 markers to assess immune cell reconstitution before mice were used experimentally.

### Flow Cytometry

Naïve or tumor-bearing lung single-cell suspensions were generated as previously described (Blake et al., 2016) and incubated with anti-CD16/32 (2.4G2) to block Fc receptors on ice prior to surface staining with the antibodies. The following antibodies were used for FACs analysis: anti-CD45.2 (104), anti-TCRβ (H57-597), anti-NK1.1 (PK136), anti-NKp46 (29A1.4), anti-CD45R (B220, RA3-6B2), anti-F4/80 (BM8), anti-CD69 (H1.2F3) (all from BioLegend, eBioscience), and MR1 tetramers. For intracellular cytokine staining, cells were surface stained as described above and then fixed and permeabilized with a cytofix/cytoperm kit (BD Biosciences) followed by staining with anti-IFNγ (XMG1.2), anti-TNF (MP6-XT22), anti-IL-17A (TC11-18H10.1) or isotype (eBio299Arm) antibody (all from BioLegend). All data were collected on a Fortessa 4 flow cytometer (BD Biosciences) and analyzed with FlowJo v10 software (TreeStar, Inc.).

### Ex-Vivo Immune Cell Cytokine Degranulation Assay

Single cell suspensions from lungs of the indicated groups were incubated in a 96-well U-bottom plate in complete RPMI 1640 media. Cells were incubated in the presence or absence of cell stimulation cocktail (PMA/ionomycin) plus protein transport inhibitors (Golgistop and Golgiplug) (1000 times dilution) for 3-4 hours as indicated. Cells were then stained for surface markers and intracellular cytokine production. CD107 staining assay was used to assess the degranulation status of immune cells. Briefly, anti-CD107a (1D4B, BioLegend) antibody was added to single cell suspension during the stimulation period before these cells were surface stained and analyzed by flow cytometry.

MAIT Cell Expansion and Sorting MAIT cells were expanded using the protocol as described by Varelias (Varelias et al., 2018). Briefly, spleens from WT or TCRδ^{-/-}, or IL17a^{-/-} mice were mashed through a 40 μm cell strainer and lysed with ACK buffer to remove red blood cells. Splenocytes were cultured in complete RPMR1640 media containing 50 ng/ml (250 U/ml) mouse IL-2 (PeproTech catalogue #212-12) and 100 nM 5-OP-RU. On day 6 and 7, cells were harvested for sorting. Anti-CD16/32 (2.4G2) to block Fc receptors were added to the single cell suspension before staining with biotin-B220 antibody (Clone RA3-6B2, Miltenyi Biotec, catalogue #130-101-928) followed by Streptavidin MicroBeads (Miltenyi Biotec, catalogue #130-048-101) to deplete B220+ cells by MACS. The enriched splenocytes were stained with an antibody cocktail and MAIT cells (defined as B220⁻ F4/80⁻ CD45.2⁺ TCRβ⁺ MR1-5-OP-RU tetramer⁺) were sorted on the Aria II/Aria III flow cytometry. MAIT cells of 85% to 95% purity were used in subsequent experiments.
MR1 knockout with CRISPR-Cas9 in B16F10 Cells

MR1 was knocked out in B16F10 cell using the CRISPR-Cas9 system. Three mouse MR1 single guide RNAs (sgRNAs) were designed on the CHOPCHOP website (http://chopchop.cbu.uib.no/). Briefly, the three MR1-sgRNAs were subcloned into PX459 vectors (Addgene, catalog #62988). PX459 plasmids containing MR1-sgRNA and pRp GFP-expressing plasmids were co-transfected into B16F10 cells using FuGENE®6 Transfection Reagent (Promega, catalog #E2691). On the following day, cells were cultured in media containing 1 μg/ml puromycin (Sigma-Aldrich) for 2 days. The GFP⁺ cells were sorted using the FACSAria III cell sorter (BD BioScience) into 96-well flat-bottom plate (1 cell/well) to obtain monoclonal B16F10-MR1^{KO} cell lines. MR1 knockout in B16F10 cells was verified by determining MR1 surface expression after 100 nM 5-OP-RU stimulation for 4 hours as described above.

### MR1 Re-Expression in B16F10-MR1 Cells

B16F10-MR1^{KO} cells were transfected with MR1-expressing pCMV6-AC-GFP plasmid (OriGene, cat #MG205125) or empty control plasmid (OriGene, catalogue #PS100010) by using FuGENE®6 Transfection Reagent. Two days later, cells were cultured in media containing 500 μg/ml Geneticin™ Selective Antibiotic (G418 Sulfate) (ThermoFisher Scientific, catalog #10131035) for 2 weeks. GFP⁺ cells were sorted to obtain a stable cell line. MR1 expression was verified by determining MR1 surface expression after 100 nM 5-OP-RU stimulation for 4 hours as described above.

### Statistics

GraphPad Prism software was used for statistical analysis. One-way ANOVA with Tukey's post-hoc test was used for multiple comparisons and Mann-Whitney test for two-group comparison and Log-Rank (Mantel-Cox) test for mouse survival. Differences between groups were considered to be statistically significant where the p-value was less than 0.05.

### Example 2. Results

### Tumor Initiation, Growth and Metastases are Suppressed in MR1^{-/-} Mice

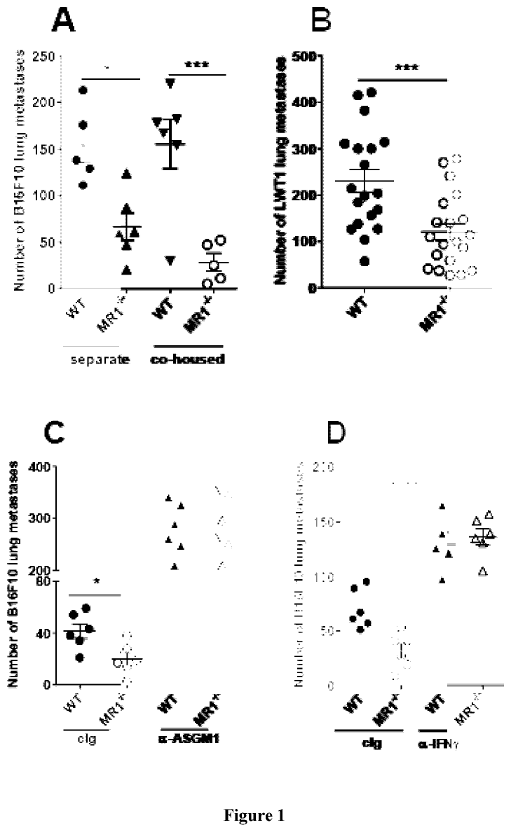

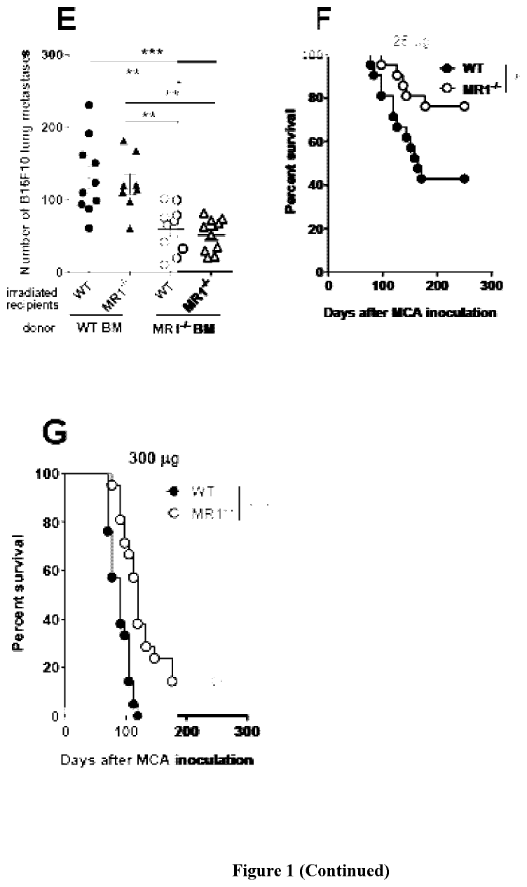

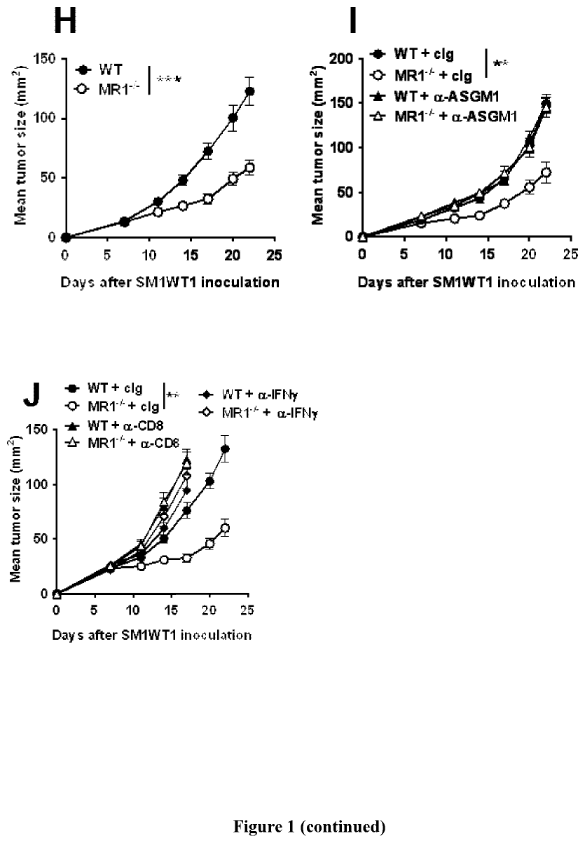

MAIT cell regulation of anti-tumor immunity has never been investigated in vivo. The role of MAIT cells in experimental tumor metastasis to the lung was investigated by comparing metastasis in C57BL/6 WT and C57BL/6 MR1^{-/-} mice, which lack MAIT cells (FIG. 1). Given that MAIT cells respond to microbial metabolites and the microbiota in C57BL/6 WT and C57BL/6 MR1^{-/-} mice are reported to be different, B16F10 melanoma cells were injected i.v. into WT or MR1^{-/-} mice which were housed separately or co-housed for up to 4 weeks prior to injection. In both settings, lung metastases were significantly reduced in MR1^{-/-} compared to WT mice, suggesting that MAIT cells promoted metastasis. The metastasis suppression in MR1^{-/-} mice was similar regardless of whether these mice were co-housed or separated from WT mice (FIG. 1A), suggesting that any decrease in lung metastases in MR1^{-/-} mice was not caused by differences in microbiota between WT and MR1^{-/-} mice. Nevertheless, to minimise any potential confounding effects of the microbiota on our experiments, co-housed WT and MR1^{-/-} mice were used in all the in vivo experiments.

LWT1, a Braf^{V600E} mutant melanoma cell line, was injected into WT and MR1^{-/-} mice to assess whether suppression of lung metastases occurred in another tumor model (FIG. 1B). Similar to B16F10, LWT1 melanoma lung metastases were significantly reduced in MR1^{-/-} compared with WT mice. Given the critical roles of NK cells and IFNγ in control of experimental lung metastases, an experiment was performed to deplete NK cells or neutralize IFNγ in B16F10 tumor-bearing WT or MR1^{-/-} mice (FIG. 1C-D). Although cIg-treated tumor-bearing MR1^{-/-} mice had fewer lung metastases compared with cIg-treated WT mice, tumor-bearing WT and MR1^{-/-} mice depleted of NK cells or neutralized of IFNγ displayed a similar higher level of lung metastases. These results demonstrated that the metastasis reduction in MR1^{-/-} mice was dependent upon NK cells and IFNγ. To determine whether loss of MAIT cells was responsible for the reduction in lung metastases, 4-way bone marrow (BM) chimeric mice were generated from WT or MR1^{-/-} donors (FIG. 1E). BM reconstitution was confirmed in the recipient mice with engraftment efficiency greater than 95% and that MAIT cells were lacking in mice transferred with MR1^{-/-} BM (data not shown). Only mice reconstituted with MR1^{-/-} BM displayed reduced tumor metastases (FIG. 1E), suggesting that loss of haematopoietic MR1 (and MAIT cell loss) contributed to lung metastases suppression in MR1^{-/-} mice. In addition to experimental lung metastases, NK cells are also required for protecting the host from MCA carcinogen-induced fibrosarcoma. Therefore, WT and MR1^{-/-} mice were injected with a low (25 μg) or high dose (300 μg) of MCA and their long-term survival (FIG. 1F, G) was monitored. At a low dose of MCA, MR1^{-/-} mice displayed greater resistance to MCA-induced fibrosarcoma than WT mice with 5/21 MR1^{-/-} mice and 12/21 WT mice developing tumors, respectively (FIG. 1F, G). Similarly, this resistance was also observed in MR1^{-/-} mice injected with a high dose of MCA compared to WT mice, suggesting that a lack of MAIT cells allowed for better protection against tumor initiation.

To determine loss of MAIT cells impacted on tumors growing outside of mucosal sites, SM1WT1 from which the LWT1 melanoma cell line was derived from, was injected subcutaneously (s.c.) into WT or MR1^{-/-} mice. Significant reduction of SM1WT1 tumor growth in MR1^{-/-} mice compared to WT mice (FIG. 1H) was observed. Furthermore, growth suppression was dependent on NK cells, CD8⁺ T cells and IFNγ as SM1WT1 tumor-bearing MR1^{-/-} mice depleted or neutralized of these cell type or cytokine, respectively, were unable to suppress tumor growth compared to cIg treated groups (FIG. 1I-J).

### MAIT Cells Promote Experimental Lung Metastases

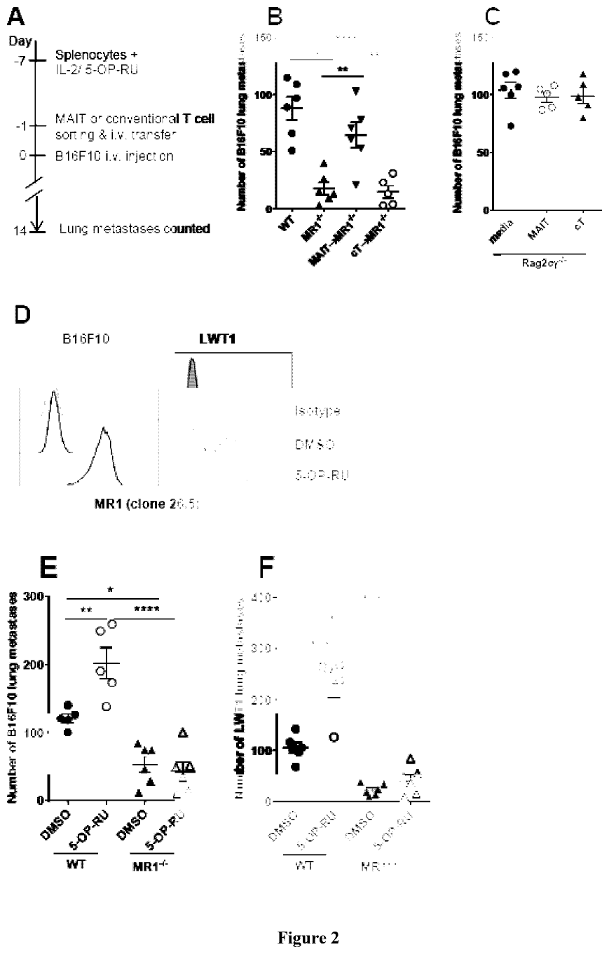

To further demonstrate that MAIT cells had a tumor-promoting function, adoptive transfer of MAIT cells into MR1^{-/-} mice was investigated to determine if it reversed the reduction in lung metastases (FIG. 2A-B). MAIT cells can be identified and sorted using MR1-tetramers. Using the protocol as indicated in the schematic (FIG. 2A), splenocytes from WT mice were cultured in the presence of MAIT cell ligand 5-OP-RU and IL-2 for 6 to 7 days. Subsequently, 2×10⁵ MAIT cells (sorted on MR1-5-OP-RU tetramer) or conventional T cells (cT, MR1-5-OP-RU tetramer negative fraction) were injected into MR1^{-/-} mice one day before B16F10 i.v. injection (FIG. 2B). While MR1^{-/-} mice had decreased lung metastases compared to WT mice, this phenotype was lost in MR1^{-/-} mice that received adoptively transferred MAIT cells, and they displayed similar numbers of lung metastases as WT mice (FIG. 2B). In contrast, transfer of conventional T cells into MR1^{-/-} mice did not reverse the observed reduction in lung metastases (FIG. 2B).

In this experiment, MAIT cells from TCRδ^{-/-} mice were also expanded, since they have a higher proportion of MAIT cells compared to WT mice, and logistically it was easier to expand sufficient MAIT cells for subsequent adoptive transfer experiments. To determine if the metastasis-promoting function of MAIT cells was mediated through direct interaction with tumor cells or indirectly through suppression of NK cells, a similar adoptive transfer experiment of MAIT cells or conventional T cells derived from TCRδ^{-/-} mice was performed into Rag2cγ^{-/-} mice that lack T, B and NK cells. In contrast to MR1^{-/-} mice, adoptive MAIT cell transfer into Rag2cγ^{-/-} mice did not further increase B16F10 lung metastases in these mice compared to the media control injected group (FIG. 2C). Similarly, conventional T cell transfer did not directly promote or suppress lung metastases. Importantly, it was demonstrated that MAIT cells expanded from TCRδ^{-/-} mice reversed the suppression of B16F10 metastases in MR1^{-/-} mice similar to WT MAIT cells (FIG. 2B). Overall, the data demonstrated that MAIT cells had a metastasis-promoting function

### Upregulation of MR1 on B16F10 Tumor Cells Increases Metastases

MR1 mRNA transcripts are present in different tissues and cell lines from mice and humans. However, surface MR1 expression on immune cells is hard to detect by flow cytometry, although its expression was upregulated and stabilized following stimulation with the MAIT cell ligand 5-OP-RU. In contrast, whether mouse tumor cell lines express MR1 has not previously been examined. Therefore, the expression of surface MR1 on a panel of eleven different mouse tumor cell lines was determined using the 26.5 clone of anti-MR1, which cross-reacts with both mouse and human MR1 (FIG. 2D). Basally, the level of MR1 was almost undetectable on the surface of all tumor cell lines examined (FIG. 2D). When these cell lines were incubated with 100 nM of 5-OP-RU and assessed for MR1 surface expression four hours later, the observed MR1 expression was substantially upregulated on the cell surface of B16F10 and LWT1 melanoma cells (FIG. 2D). MR1 expression was also upregulated to a varying extent on MC38 parental or OVA expressing colorectal adenocarcinoma cells, HcMel3, HcMel12, SM1WT1 melanoma cells and MCA1956 fibrosarcoma cells. However, MR1 was not upregulated on RM-1 prostate carcinoma cells, 4T1.2 mammary carcinoma cells or 3LL lung carcinoma cells. MR1 upregulation was assessed at 100 nM concentration and four-hour time point since it was demonstrated in a separate dose titration and time kinetic experiments using B16F10 that these conditions optimally upregulated surface MR1 on these cells (as little as 10 nM ligand upregulated MR1). The kinetics of MR1 upregulation on B16F10 cells was similar to what was previously reported for human C1R cells (B-cell lymphoblastoid cell line) (McWilliam et al., 2016). Similarly, MR1 was generally negative or very low across a range of different human cell lines which became upregulated following incubation with 5-OP-RU (not shown). Next, the impact of up-regulation of MR1 on B16F10 or LWT1 prior to i.v injection to WT or MR1−/− mice on the number of lung metastases was assessed. A significantly increased number of lung metastases in WT mice injected with 5-OP-RU-treated was observed compared to DMSO control-treated B16F10 cells and LWT1 cells and this increase was lost when tumor cells were injected into MR1^{-/-} mice (FIG. 2E, F). RM-1 which does not upregulate MR1, displayed a similar number of metastases when injected into WT or MR1^{-/-} mice. Overall, these results show that mouse tumor cell lines that are capable of expressing surface MR1 may activate MAIT cells to mediate their suppressive function.

### MAIT Cells Promote Metastases by Suppressing NK Cell Effector Function

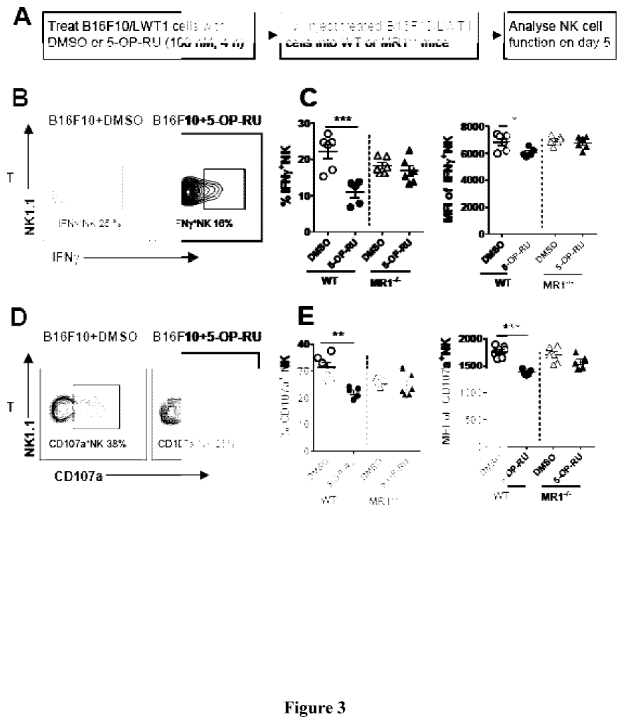

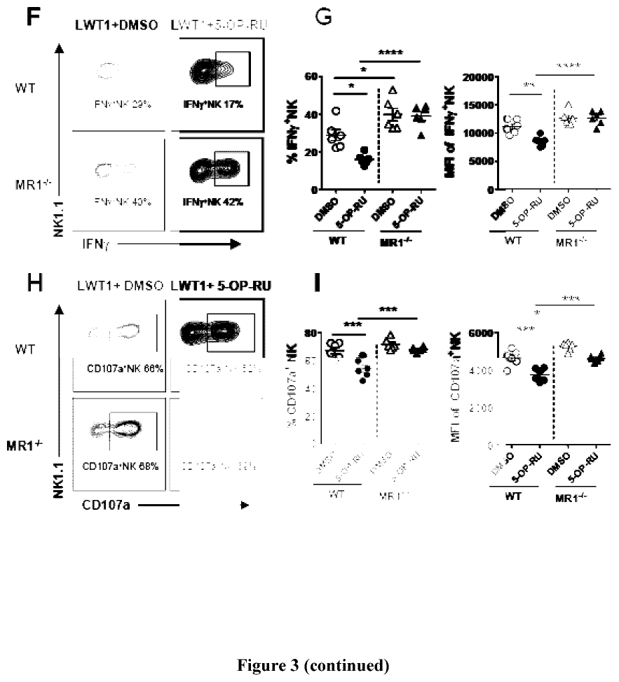

The consequence of MR1 upregulation on B16F10 on NK cell effector function was investigated (FIG. 3). B16F10 cells were stimulated with 5-OP-RU or DMSO for 4 hours, before the cells were washed and then i.v. injected into WT mice. On day 5, lungs were harvested and NK cell effector functions as measured by IFNγ production and degranulation (CD107a) were assessed (FIG. 3A). The proportions of NK cells producing IFNγ and their expression levels as measured by mean florescence intensity (MFI) were significantly decreased in the lungs of mice bearing 5-OP-RU-treated compared to DMSO-treated B16F10 cells (FIG. 3B-C). In contrast, NK cell function was not suppressed in the lungs of MR1^{-/-} mice challenged with 5-OP-RU-treated B16F10 cells compared with DMSO-treated B16F10 cells (FIG. 3B-C). Similarly, the proportions of NK cells expressing CD107a and their expression levels were also significantly decreased in the lungs of mice injected with 5-OP-RU-treated B16F10 cells compared to DMSO-treated B16F10 cells (FIG. 3D-E). A similar suppression of NK cell effector function was observed when the lungs of mice injected with B16F10 cells treated with two different doses of 5-OP-RU were harvested and analysed at a later time point on day 11. In WT mice that received 5-OP-RU-treated LWT1, suppression of NK cell effector function was observed but this did not manifest in MR1^{-/-} mice (FIG. 3F-I). An increased proportion of NK cells producing IFNγ derived from the lungs of MR1^{-/-} mice injected with DMSO-treated LWT1 was also observed when compared to similarly injected WT mice. Overall, these data show that the interaction of MAIT cells with MR1-expressing B16F10 or LWT1 tumor cells played a role in suppressing the anti-metastatic activity of NK cells.

Increased Production of IL-17A and TNF in MAIT Cells Derived from the Lungs of Mice Injected with 5-OP-RU-Treated B16F10 Cells

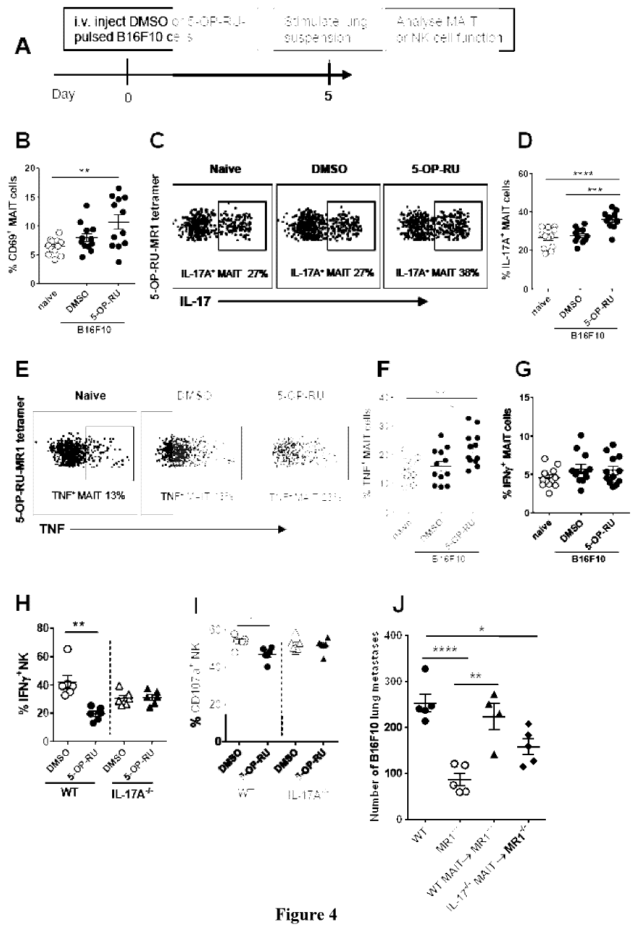

As MAIT cells can rapidly secrete effector cytokines, it was determined whether their activation status and cytokine profile were attenuated in the lungs of mice injected with 5-OP-RU- or DMSO-treated B16F10 tumor-bearing mice (FIG. 4A). CD69 is a commonly used marker to determine MAIT cell activation status and tissue residency. In this study, an increased proportion of CD69⁺ MAIT cells that expressed CD69, IL-17A and TNF in the lungs of mice injected with 5-OP-RU- compared to DMSO-treated B16F10 tumor cells was observed (FIG. 4B-F). In contrast, the proportion of MAIT cells producing IFNγ did not change (FIG. 4G). Given the increase in the proportion of IL-17-producing MAIT cells, the question was asked how the loss of IL-17A impacted on NK cell effector function. In an experimental setup similar to FIG. 3, WT or IL-17A^{-/-} mice were injected with 5-OP-RU- or DMSO-treated B16F10 cells and 5 days later lungs were harvested for NK cell analysis. Although the proportions of IFNγ producing and CD107a expressing NK cells were significantly reduced in WT mice, as demonstrated earlier, this reduction was not observed in IL-17A^{-/-} mice (FIG. 4H-I), suggesting that IL-17A may be one mechanism by which the anti-metastatic activity of NK cells are suppressed. To confirm the IL-17A produced by MAIT cells was required for its suppressive effect, MAIT cells from adoptively transferred from WT or IL-17A^{-/-} mice into MR1^{-/-} mice bearing B16F10 lung metastases (FIG. 4J). MAIT cells that were unable to produce IL-17A were less effective at reversing the suppressive phenotype compared to WT MAIT cells suggesting IL-17A was partially required for suppression In vitro co-culture of purified splenic MAIT cells with 5-OP-RU compared to DMSO-treated B16F10 cells produced more IFNγ, IL-17A and TNF. This response was specific as no increase in cytokine production was observed in conventional T cells cultured with 5-OP-RU compared to DMSO treated B16F10. Similar to previous reports that human MAIT cells displayed cytotoxic capability, a decrease in the number of 5-OP-RU-treated CTV-labeled B16F10 cells following co-culture with MAIT cells was observed but not with conventional T cells. These data shows that the anti-tumor phenotype of MAIT cells observed in vitro may not reflect their physiological role in vivo.

Loss of Surface MR1 on B16F10 Cells Decreased their Metastatic Potential in WT Mice

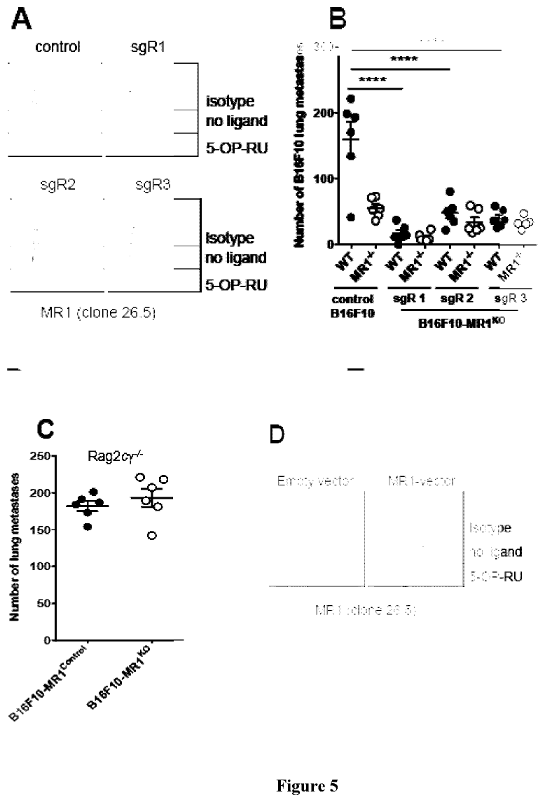

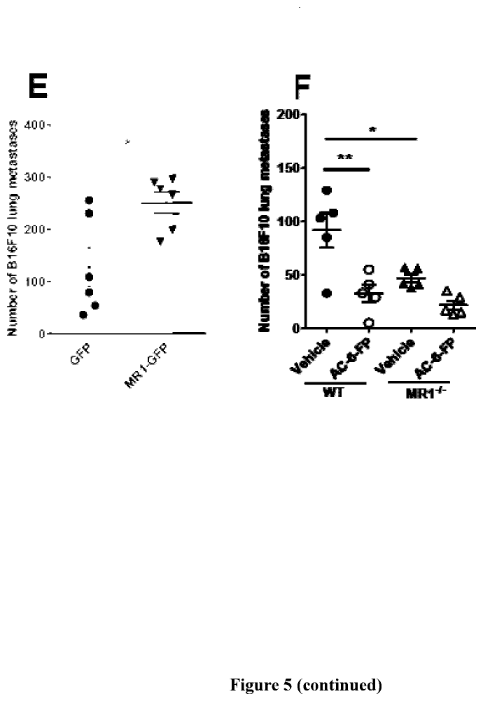

The data herein shows the importance of MR1 expression on B16F10 cells to activate the suppressive function of MAIT cells. Using CRISPR/Cas9, three different short-guide RNAs (sgRNAs) targeting the mouse MR1 gene were designed and transfected into B16F10 cells, respectively, while transfection of B16F10 cells with an empty vector served as a control (FIG. 5). As shown in FIG. 5A, all three sgRNAs effectively knocked out MR1 in B16F10 cells (B16F10-MR1^{KO}) compared to vector control transfected B16F10 tumor cells. Using in vitro assays, it was confirmed that loss of MR1 in B16F10 did not intrinsically affect their biology no changes in their proliferation or migration ability were observed. Similarly, co-culture of B16F10 with 5-OP-RU did not alter the biology of these cells as measured by their proliferative or migratory assays. Next, the three B16F10-MR1^{KO} or vector control B16F10 tumor cell lines were injected intravenously into WT and MR1^{-/-} mice and their lung metastases burden was determined 14 days later (FIG. 5B). The number of metastases was dramatically reduced in WT mice injected with the B16F10-MR1^{KO} cell lines compared to those that received vector control B16F10 (FIG. 5B). Furthermore, for all three B16F10-MR1^{KO} cell lines, no further decrease in metastases in the MR1^{-/-} mice compared to WT mice was observed (FIG. 5B). It was demonstrated that this decrease in metastases was due to the loss of MR1 and not caused by the transfection process as the number of metastases was similar in WT mice injected with parental or vector control B16F10. In vivo, it was confirmed that the loss of MR1 on B16F10 did not intrinsically impact on their ability to form lung metastases as similar numbers were observed between Rag2cγ^{-/-} mice i.v. injected with B16F10 vector control or B16F10-MR1^{ko} (sgR3) (FIG. 5C). Finally, when MR1 was over-expressed in B16F10-MR1^{ko} (sgR3) cells, it was again shown that MR1 could be upregulated on their cell surface following co-culture with 5-OP-RU (FIG. 5D). Injection of B16F10 over-expressing MR1 into WT mice increased the number of metastases compared to control vector-transfected B16F10-MR1^{ko} (sgR3) cell line without having to pre-treat them with 5-OP-RU (FIG. 5E). To determine if activation of MAIT cells was required for their suppression of NK cells, AC-6-FP or vehicle-treated B16F10 were injected into WT or MR1^{-/-} (FIG. 5F). AC-6-FP upregulates MR1, but does not activate MAIT cells. A decrease in metastases numbers in WT mice injected with AC-6-FP-treated compared to vehicle-treated B16F10 tumor cells was observed. This level of reduction in metastases was similar to MR1^{-/-} mice injected with AC-6-FP-treated or vehicle-treated B16F10 cells (FIG. 5F). These data show that some ligands may be used to block the normal activation signal that MAIT cells are receiving via MR1 in the tumor microenvironment (TME). Overall, the data shows the potential for tumors to upregulate MR1 and activate MAIT cell immunoregulatory function.

### Blockade of MR1 Suppresses Experimental Lung Metastases and Osteosarcoma

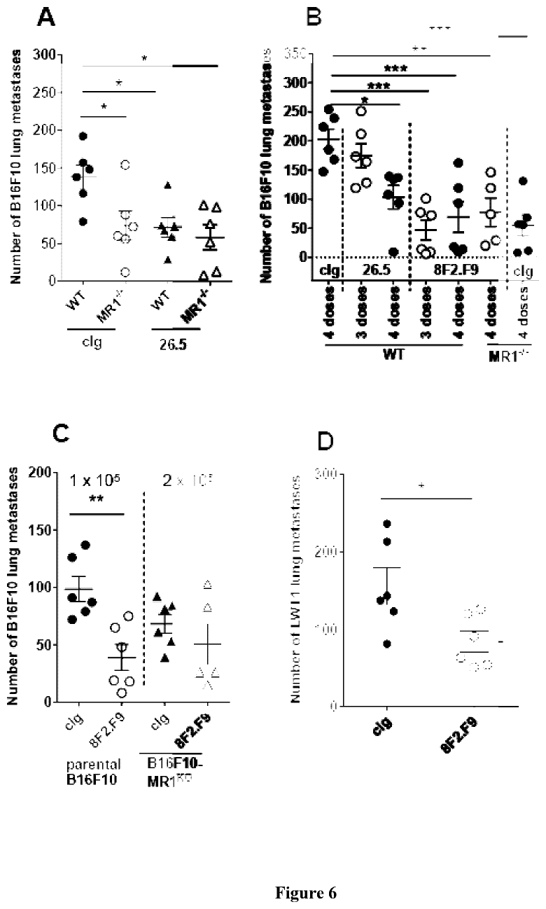

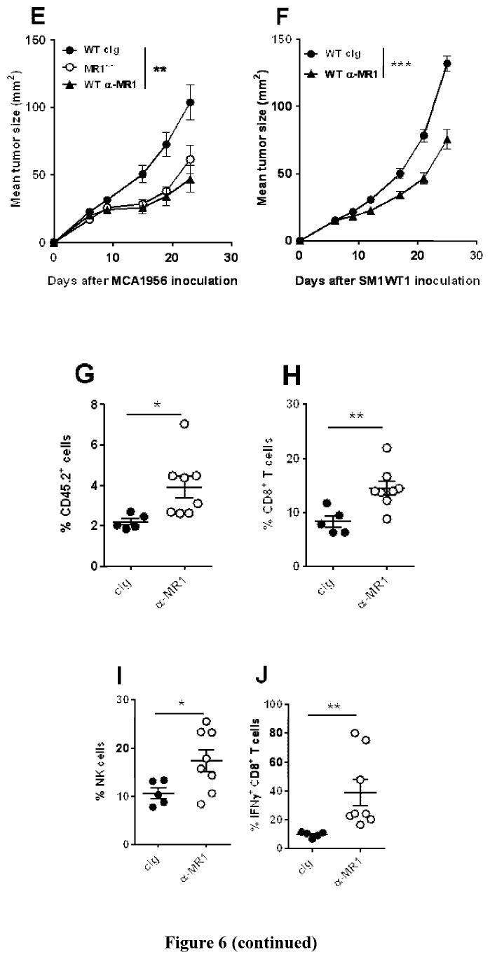

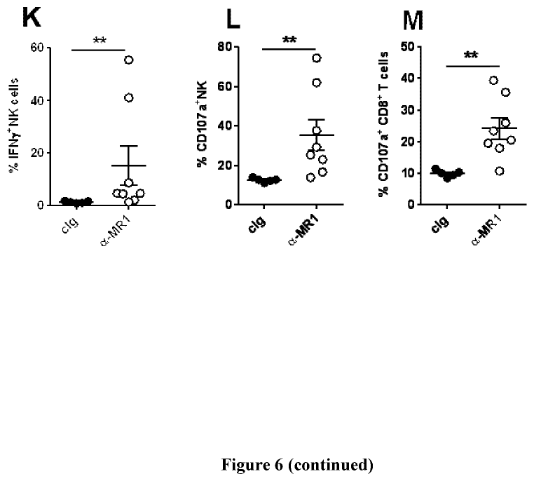

In addition to their use in flow cytometry analysis, the 26.5 and 8F2.F9 clones of anti-MR1 are known to block MAIT cells from interacting with MR1. Therefore, it was asked if the effect observed in MR1^{-/-} mice was recapitulated with these MR1 blocking antibodies (FIG. 6). MR1 blockade by clone 26.5 on days −1, 0 and 3 and 7, relative to B16F10 i.v injection, effectively suppressed lung metastases to a similar level as observed in MR1^{-/-} mice (FIG. 6A). Furthermore, the specificity of antibody 26.5 was confirmed, as no further decrease in the number of lung metastases was observed between tumor-bearing MR1^{-/-} mice treated with 26.5 or cIg (FIG. 6A). Next, a comparison was made of whether giving three or four doses of clone 26.5 or 8F2.F9 (days −1, 0, 3 vs days −1, 0, 3 and 7) equivalently suppressed metastases (FIG. 6B). Interestingly, suppression of lung metastases was more effective when four doses of 26.5 were given compared to three doses (FIG. 6B). In contrast, administering three or four doses of 8F2.F9 equivalently reduced B16F10 lung metastases and this suppression appeared to be superior to that observed using the 26.5 clone (FIG. 6B). The specificity of the 8F2.F9 clone for MR1 was confirmed, as no further decrease in number of lung metastases was observed between tumor-bearing MR1^{-/-} mice treated with 8F2.F9 or cIg (FIG. 6B). The lack of any further decrease in lung metastases in MR1^{-/-} mice treated with 26.5 or 8F2.F9 also suggested that these antibodies probably do not directly impact on B16F10 metastasis (FIG. 6A-B). Furthermore, the utility of blocking MR1 on tumors was demonstrated, since B16F10-MR1^{ko} (sgR3) lung metastases numbers were not reduced between 8F2.F9- and cIg-treated mice compared to similar groups of treated mice bearing B16F10 parental metastases (FIG. 6C). Finally, it was shown that 8F2.F9 treatment also significantly reduced LWT1 lung metastases compared with cIg treatment (FIG. 6D).

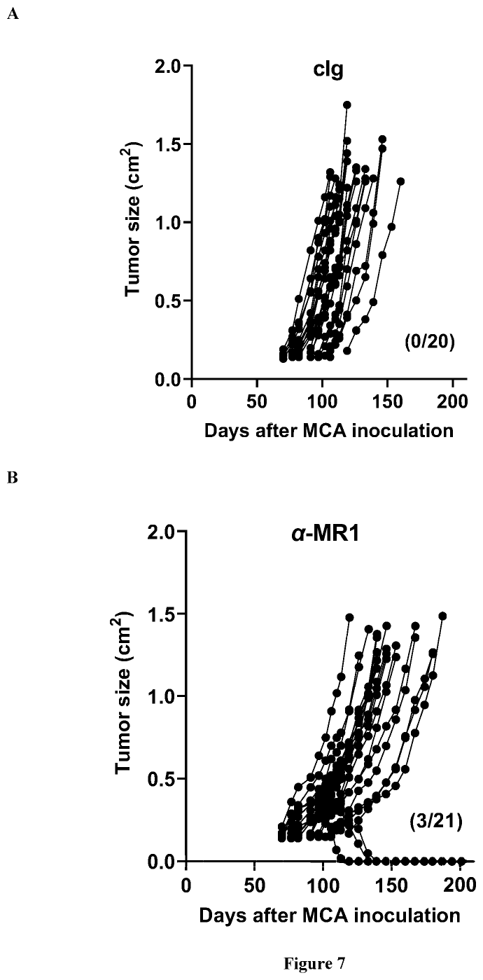

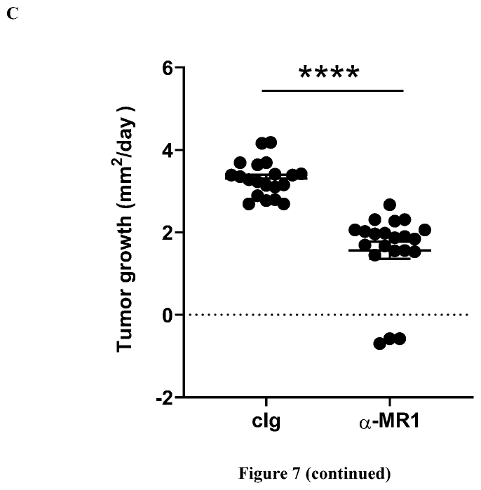

In mice bearing established s.c. MCA1956 or SM1WT1 tumors (FIG. 6E, F), treatment with 4 doses of anti-MR1 significantly suppressed tumor growth compared to cIg treated groups. The therapeutic potential of anti-MR1 therapy was also assessed using established de novo MCA-induced fibrosarcomas (FIGS. 7A, B and C).

Although cIg-treated fibrosarcomas grew rapidly and all mice succumbed to their tumors (FIG. 7A), anti-MR1 reduced the growth of most tumors (FIG. 7B, C) and caused the complete rejection of three of 21 tumors.

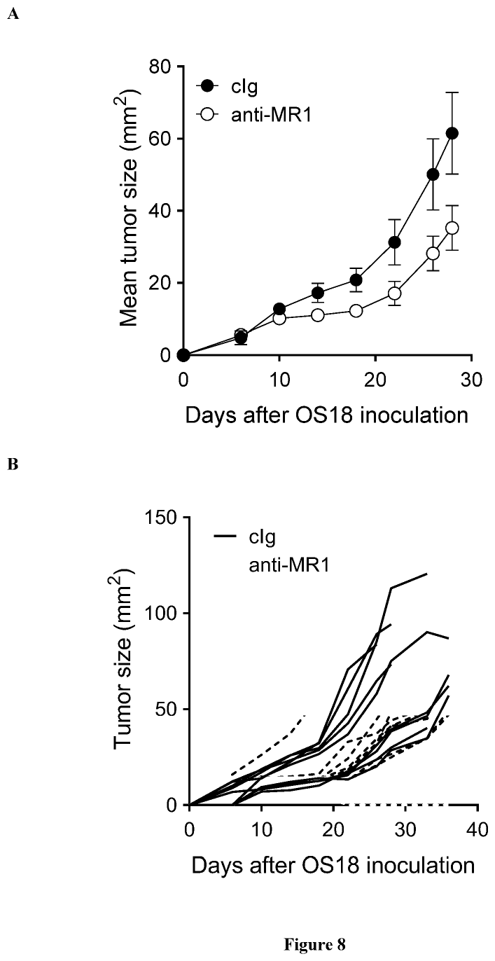

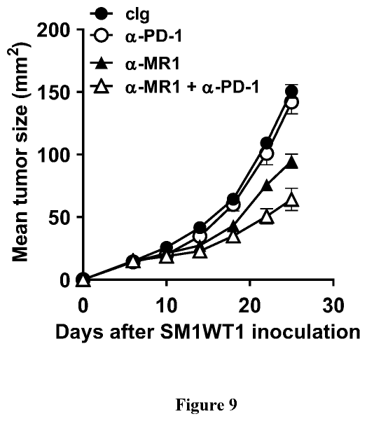

In mice bearing established s.c OS18 osteosarcoma tumors (FIG. 8), anti-MR1 suppressed tumor growth compared to cIg treated groups.

FACs analysis was performed on the tumor-infiltrating lymphocytes (TILs) on endstage SM1WT1 tumors from FIG. 6F. Interestingly, a significant increase was observed in the proportion of total CD45⁺ immune cells (FIG. 6G), CD8⁺ T cells (FIG. 6H) and NK cells (FIG. 6I) in the anti-MR1 compared to cIg treated groups. Furthermore, there was an increased proportion of IFNγ producing and CD107a expressing CD8⁺ T cells and NK cells from the TILs of anti-MR1 compared to cIg-treated mice following restimulation with PMA/Ionomycin (FIG. 6J-M). Overall, these data suggested that the interaction of MR1 on tumors and MAIT cells might be critical in suppressing anti-tumor immunity in mucosal and non-mucosal sites and blocking MR1 may represent a new strategy for cancer immunotherapy.

Discussion

In this study, it was demonstrated for the first time that MAIT cells promoted tumor initiation, growth and metastases. Using two experimental mouse models of lung metastases, a significant decrease in the number of lung metastases was observed in MR1^{-/-} compared to WT mice and this reduction was also seen using two different MR1 blocking antibodies. In addition, MR1^{-/-} mice were more resistant to carcinogen-induced fibrosarcoma development compared to WT mice. Furthermore, it was demonstrated that loss of MAIT cells impacted on tumors growing outside of mucosal sites as MCA1956 and SM1WT1 s.c tumor growth was suppressed both in MR1^{-/-} mice and in WT mice treated with anti-MR1. By performing adoptive transfer of purified MAIT cells into B16F10 tumor-bearing MR1^{-/-} mice, the reduction in lung metastases in these mice was reversed, whereas conventional T cell transfer was without effect. It was also demonstrated that MAIT cells promoted lung metastases by engaging MR1 on tumor cells resulting in an IL-17A-dependent suppression of NK cell effector function. The importance of surface MR1 on tumor cells was also demonstrated by the increased number of metastases in mice injected with 5-OP-RU pre-treated B16F10 tumor cells. In contrast, deletion of MR1 on B16F10, or pre-treatment of B16F10 with Ac-6-FP, dramatically reduced the number of lung metastases, while re-expression of MR1 reversed this phenotype. These data sit in contrast with the previous dogma that MAIT cells were directly cytotoxic to tumor cells, rather illustrating in vivo that MAIT cells act to suppress NK cell effector function and thereby promote tumor metastasis. This is a new concept in that NK cells were thought, prior to the teachings of the present specification, to be suppressed only by Treg, or myeloid/granulocyte populations, or by MHC class I binding tumor itself. Described herein is an important MHC class I like recognition system that can also impact on NK cell effector function.

Therapeutically, two different clones of MR1 blocking antibodies (26.5, 8F2.F9) were as capable of reducing B16F10 and LWT1 lung metastases as loss of MAIT cells in MR1^{-/-} mice. Other experiments showed that their anti-metastatic effect was mediated through blocking MAIT cell activation rather than having a direct anti-tumor effect on MR1-expressing tumor cells. Anti-MR1 alone also suppressed s.c tumor growth of MCA1956 and SM1WT1 suggesting that the MR1-MAIT pathway operates in tumors growing in mucosal or non-mucosal sites. SM1WT1 generally does not respond to PD1 blockade. When end-stage SM1WT1 tumors were collected for flow cytometry analysis, a significant increase was observed in the proportion of CD8⁺ T and NK cells in the TILs from anti-MR1 compared to cIg treated groups. Furthermore, an increased proportion of these CD8⁺ T and NK cells displayed improved effector function as observed by expression of IFNγ and CD107a. Given that MR1 is highly conserved across 150 million years of mammalian evolution, blockade of the tumor MR1-MAIT cell axis in humans provides an approach to relieve immune-mediated suppression of NK and CD8⁺ T cells in the TME. Future experiments and combination approaches can now be used to try and boost NK cell and CD8⁺ T cell anti-tumor activity by blocking tumor MR1 in tumors where MR1 is highly expressed. For example, blocking MR1 on these tumors might allow better retention of NK/CD8⁺ T cells and their effector functions in the context of immune checkpoint blockade or other approaches that reduce myeloid-mediated immunosuppression. Overall, our data shows that MAIT cells suppress NK cell anti-metastatic function and that blocking MR1 or saturating it with an inhibitory ligand provides a new therapeutic strategy for cancer immunotherapy.

### Example 3. Anti-PD1+Anti-MR1 Combination Therapy

Groups of C57BL/6 WT mice (n=7-9/group) were injected s.c. with 1×10⁶ SM1WT1 melanoma cell. Mice were i.p. treated with 250 μg cIg (1-1) or anti-MR1 (clone 8F2.F9) or anti-PD1 (RMP1-14) or a combination of anti-MR1 and anti-PD1 on days 6, 10, 14 and 18 relative to tumor inoculation. Mice were monitored for tumor growth (calculated by the product of two perpendicular axes). Significant differences between the indicated groups were determined using one-way ANOVA followed by Tukey post-hoc test, *p<0.05, ****p<0.0001.

Mice treated with a combination of anti-MR1 and anti-PD1 had a decreased mean tumour size compared to mice treated with anti-MR1 alone.

### Example 4. MR1 Expression on Melanoma and Colorectal Cancers

### Immunohistochemistry

Archival-FFPE tumors were sectioned at 4 μm on superfrost+ slides. Slides were deparaffinised in xylene, and rehydrated in graded ethanol. Antigen retrieval was performed in DAKO target retrieval solution (Citrate pH6) (Perkin Elmer; AR600250ML) in a Decloaking Chamber (Biocare Medical) at 100° C. for 20 minutes. IHC was performed on an Autostainer-Plus (DAKO). Primary antibodies against MR1 (Abcam; ab229715) were incubated for 45 minutes at room temperature using a 1:400 dilution in Da Vinci Green (BioCare Medical; PD900). Staining was visualized using a MACH3 Rabbit HRP Polymer detection system (BioCare Medical; M3R531L) and DAB Chromogen Kit (BioCare Medical; BDB2004L) and counterstained with diluted hematoxylin. Using sequentially cut slides that were stained for MR1, an appropriate tumour marker (SOX10 for melanoma) or a H&E stained slide (for CRC), the tumour cells within the sample were identified and assessed for the level of MR1 expression. MR1+ tumor cells were assigned as follows: score 0+ (negative), score 1+<10%, score 2+10-50%, score 3+>50%.

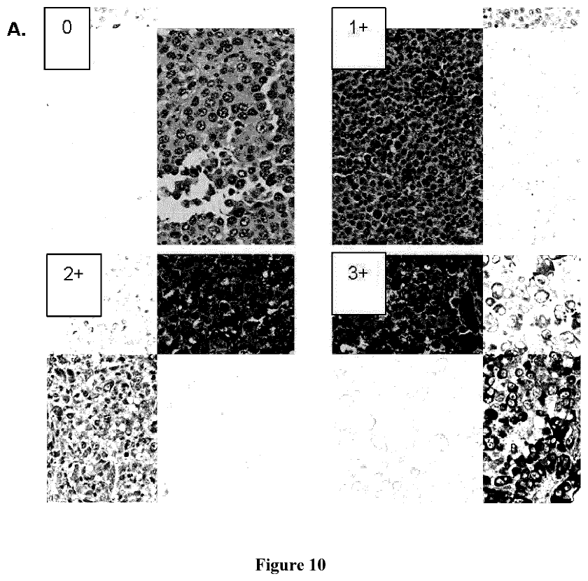

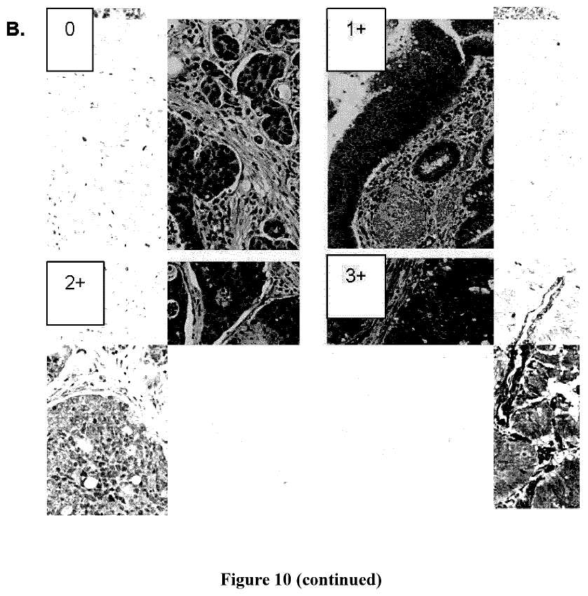

Using immunohistochemistry, MR1 expression was assessed in (A) melanoma and (B) colorectal carcinoma. The samples were scored on their tumor cell expression of MR1; SOX10+(melanoma) and H&E (CRC) staining was used to verify tumor cells. All samples were stained using Abcam polyclonal antibody 229715. Object lens 20×. In FIG. 10A Melanoma samples 0, 1+, 2+ and 3+ are a stage IV skin metastasis, stage III lymph node metastasis, stage IV lung metastasis and stage III brain metastasis, respectively. In FIG. 10B. CRC samples are all stage IV; 0, 1+, 2+ and 3+ are a lymph node metastasis, primary tumor, visceral metastasis and primary tumor, respectively.

As illustrated, MR1 can be detected on melanoma and CRC tumor cells.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Armour K L. et al., 1999 Eur J Immunol. 29(8):2613-24
Blake et al. (2016) Cancer Discov, 6:446-59
Corbett et al. (2014) Nature, 509:361-5
Duncan et al., (1988) Nature 332:563
Ferrari de Andrade et al. (2014) Cancer Res, 74:7298-308
Godfrey et al. (2019) Nat Immunol, 20:1110-1128
Huang et al. (2005) J Biol Chem, 280(22):21183-93
Kansara et al. (2019) Cancer Discov. 9:1511-1519
Kawachi et al. (2006) J Immunol, 176(3):1618-27
Le Bourhis et al. (2011) Trends Immunol, 2011; 32:212-8
McWilliam et al. (2016) Nat Immunol, 17:531-7
Mittal et al. (2019) Cancer Immunol Res, 7:559-571
Tilloy F et al. (1999) J Exp Med, 189:1907-21
Treiner et al. (2003) Nature 422(6928): 164-9
Treiner et al. (2005) Microbes Infect, 7(3):552-9
Varelias et al. (2018) The Journal of Clinical Investigation, 128
Yan et al. (2018) Cancer Immunol Res, 6:978-87
Yan et al. (2020) Cancer Discov 10:124-141

1. A method of treating cancer in a subject, the method comprising administering to the subject a compound that inhibits MR1-mediated MAIT cell activation.

2. A method of treating cancer in a subject, the method comprising administering a compound that binds to MR1 on tumor cells in the subject.

3. The method of claim 1 or claim 2, wherein the compound binds to MR1 and inhibits MR1-mediated MAIT cell activation.

4. A method of increasing the response of tumor cells in a subject to cancer therapy, the method comprising administering to the subject a compound that binds to MR1 on tumor cells in the subject.

5. The method of claim 4, wherein the cancer therapy is immunotherapy or targeted therapy.

6. The method of claim 5, wherein the cancer immunotherapy is selected from antibody therapy, CAR-T cell therapy, immune checkpoint inhibitor therapy, and/or cytokine therapy.

7. The method of any one of claims 1 to 6, wherein the compound binds to MR1 and inhibits MR1 signalling of MAIT cells.

8. The method of claim 7, wherein the compound that binds MR1 is ligand that inhibits MR1 signalling of MAIT cells.

9. The method of any one of claims 1 to 8, wherein the compound that binds to MR1 is an antibody.

10. The method of claim 9, wherein the antibody is a multivalent antibody.

11. The method of claim 10, wherein the multivalent antibody is a bispecific antibody.

12. The method of claim 10 or claim 11, wherein the multivalent antibody binds to an immune checkpoint molecule, T-cell surface molecule, and/or an NK cell surface molecule.

13. The method of claim 12, wherein the immune checkpoint molecule is selected from PD-1, PD-L1, CTLA-4, A2AR, CD73, B7-H3, B7-H4, BTLA, IDO, KIR, LAG3, NOX2, TIM-3, VISTA, CD39, TIGIT, CD96, CD155, IL23R and SIGLEC7.

14. The method of any one of claims 1 to 13, wherein administering a compound that inhibits MR1-mediated MAIT cell activation and/or that binds to MR1 on cancer cells in a subject increases T cell and/or NK cell tumor infiltration in the subject.

15. The method of any one of claims 1 to 14, wherein the method is performed in conjunction with an additional cancer therapy.

16. The method of claim 15, wherein the additional cancer therapy is selected from radiotherapy, surgery, targeted therapy, and/or chemotherapy.

17. The method of claim 16, wherein the additional cancer therapy is an anti-PD1 antibody.

18. A method of determining the likelihood of cancer metastasis in a subject, the method comprising:
(a) detecting the level of expression of MR1 in tumor cells in the subject, and
(b) comparing the level of expression of MR1 in tumor cells in the subject to a reference level of expression of MR1 on cancer cells,
wherein a higher level of expression of MR1 in tumor cells in the subject compared to the reference level of expression of MR1 in tumor cells is indicative of the patient having an increased risk of cancer metastasis.

19. The method of claim 18, wherein the reference level of expression of MR1 in cancer cells is derived from a control sample, a normal reference sample and/or a predetermined level of MR1 expression on cancer cells.

20. The method of claim 18 or claim 19, wherein the method comprises obtaining a sample comprising cancer cells from the subject and detecting the level of expression of MR1 in the cancer cells.

21. The method of any one of claims 18 to 20, wherein the method comprises detecting MR1 polypeptide on the cell surface of the cancer cells.

22. The method of any one of claims 18 to 21, wherein the method comprises contacting the cancer cells with a compound that binds to MR1 and detecting the compound bound to MR1.

23. The method of claim 22, wherein the compound that binds to MR1 is an antibody.

24. A method of selecting a patient for treatment with a compound that binds MR1 on tumor cells, the method comprising determining whether MR1 is expressed on tumor cells in the patient, wherein a patient is selected for treatment with a compound that binds MR1 on the basis of MR1 expression on the cancer cells.

25. A method of determining a likelihood of a positive or negative clinical response in a patient to a cancer therapy, the method comprising comparing the level of expression of MR1 on tumor cells in the subject to a reference level of expression of MR1 on tumor cells,
wherein a higher level of expression of MR1 on tumor cells in the subject compared to the reference level of expression of MR1 on tumor cells is indicative of the patient having an increased likelihood of a positive clinical response to the cancer therapy, wherein the cancer therapy comprises administration of compound that inhibits MR1-mediated MAIT cell activation and/or that binds to MR1 on tumor cells in the patient.

26. The method of claim 25, wherein the method comprises detecting the level of expression of MR1 on tumor cells in the subject.

27. A method of preventing or reducing the likelihood of cancer metastasis in a subject, the method comprising administering to the subject a compound that binds to MR1 on tumor cells in the subject.

28. The method of any one of claims 1-27, wherein the cancer is selected from the group consisting of: lung cancer, non-small-cell lung carcinoma, small-cell lung carcinoma, fibrosarcoma, colorectal carcinoma and osteosarcoma.


SEQUENCE LISTING (21 sequences)

SEQ ID NO: 1 - protein, 341 aa, Homo sapiens
MGELMAFLLPLIIVLMVKHSDSRTHSLRYFRLGVSDPIHGVPEFISVGYVDSHPITTYDSVTRQKEPRAPWMAENLAPDHWERYTQLLRGWQQMFKVELKRLQRHYNHSGSHTYQRMIGCELLEDGSTTGFLQYAYDGQDFLIFNKDTLSWLAVDNVAHTIKQAWEANQHELLYQKNWLEEECIAWLKRFLEYGKDTLQRTEPPLVRVNRKETFPGVTALFCKAHGFYPPEIYMTWMKNGEEIVQEIDYGDILPSGDGTYQAWASIELDPQSSNLYSCHVEHCGVHMVLQVPQESETIPLVMKAVSGSIVLVIVLAGVGVLVWRRRPREQNGAIYLPTPDR

SEQ ID NO: 2 - DNA, 417 bp, Mus musculus
atggaatggagcagagtctttatctttctcctatcaataactgcaggtgttcactcccaggtccagctgcagcagtctggaactgagctggtaaggcctgggacttcagtgaaggtgtcctgcaaggcttctggatacgccttcagtaattacctgatagaatggataaagcagaggcctggacagggccttgagtggattggagtgattaatcctggaagtggtggtattagctacaatgagaagttcaagggcaaggcaacactgactgcagacaaatcctccagcactgcctacatgcagctcagcagcctgacatctgaggactctgcggtctatttctgtgcaagatggggttatctatattactatactatggaccactggggtcaaggaacctcagtcaccgtctcctca

SEQ ID NO: 3 - DNA, 393 bp, Mus musculus
atgaagttgcctgttaggctgttggtgctgatgttctggattcctgcttccagcagtgatgttttgatgacccaaactccactctctctgcctgtcagtcttggagatcaagcctccatctcttgtagatctagtcagagcattgtacagggtagtggaaacacctttttagattggtacttacagaaaccaggccagtctccaaaggtcctgatctacaaagtttccaaccgattttctggggtcccagccaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 4 - protein, 139 aa, Mus musculus
MEWSRVFIFLLSITAGVHSQVQLQQSGTELVRPGTSVKVSCKASGYAFSNYLIEWIKQRPGQGLEWIGVINPGSGGISYNEKFKGKATLTADKSSSTAYMQLSSLTSEDSAVYFCARWGYLYYYTMDHWGQGTSVTVSS

SEQ ID NO: 5 - protein, 5 aa, Mus musculus
NYLIE

SEQ ID NO: 6 - protein, 17 aa, Mus musculus
VINPGSGGISYNEKFKG

SEQ ID NO: 7 - protein, 11 aa, Mus musculus
WGYLYYYTMDH

SEQ ID NO: 8 - protein, 131 aa, Mus musculus
MKLPVRLLVLMFWIPASSSDVLMTQTPLSLPVSLGDQASISCRSSQSIVQGSGNTFLDWYLQKPGQSPKVLIYKVSNRFSGVPARFSGSGSGTDFTLKISRVEAEDLGVYYCFQGSHVPYTFGGGTKLEIK

SEQ ID NO: 9 - protein, 16 aa, Mus musculus
RSSQSIVQGSGNTFLD

SEQ ID NO: 10 - protein, 7 aa, Mus musculus
KVSNRFS

SEQ ID NO: 11 - protein, 9 aa, Mus musculus
FQGSHVPYT

SEQ ID NO: 12 - DNA, 411 bp, Mus musculus
atgaaattcagctgggtcatcttcttcctgatggcagtggttacaggggtcaattcagaggttcagctgcagcagtctgtggcagagcttgtgaggccaggggcctcagtcaagttgtcctgcacagcttctggcttcaacattaaaaatacctatatgcactgggtgaagaagaggcctgaactgggcctggagtggattggaaggattgatcctgcgaatggtaatactaattttgccccgaagttccagggcaaggccactataactgcagacacatcctccaacacagcctacctgcagctcagcagcctgacatctgaggacactgccatctattactgtcctagagggagtggtaattactactttgactactggggccaaggcaccactctcacagtctcctca

SEQ ID NO: 13 - DNA, 378 bp, Mus musculus
atgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggacctgttaaactcctgatctactatacatcaagattacactcaggagtcccatcaaggttcagtggcagtgggtctggaacagattattctctcaccattaacaacctggcgcaagaagatattgccacttacttttgccaacagggtaatacgcttccttacacgttcggaggggggaccaagctggaaataaaa

SEQ ID NO: 14 - protein, 137 aa, Mus musculus
MKFSWVIFFLMAVVTGVNSEVQLQQSVAELVRPGASVKLSCTASGFNIKNTYMHWVKKRPELGLEWIGRIDPANGNTNFAPKFQGKATITADTSSNTAYLQLSSLTSEDTAIYYCPRGSGNYYFDYWGQGTTLTVSS

SEQ ID NO: 15 - protein, 5 aa, Mus musculus
NTYMH

SEQ ID NO: 16 - protein, 17 aa, Mus musculus
RIDPANGNTNFAPKFQG

SEQ ID NO: 17 - protein, 9 aa, Mus musculus
GSGNYYFDY

SEQ ID NO: 18 - protein, 126 aa, Mus musculus
MSSAQFLGLLLLCFQGTRCDIQMTQTTSSLSASLGDRVTISCRASQDISNYLNWYQQKPDGPVKLLIYYTSRLHSGVPSRFSGSGSGTDYSLTINNLAQEDIATYFCQQGNTLPYTFGGGTKLEIK

SEQ ID NO: 19 - protein, 11 aa, Mus musculus
RASQDISNYLN

SEQ ID NO: 20 - protein, 7 aa, Mus musculus
YTSRLHS

SEQ ID NO: 21 - protein, 9 aa, Mus musculus
QQGNTLPYT